(19) United States
(12) Patent Application Publication
(10) Pub. No.: US 2023/0416812 A1
(43) Pub. Date: Dec. 28, 2023

(54) METHOD CAPABLE OF MAKING ONE CLUSTER BY CONNECTING INFORMATION OF STRANDS GENERATED DURING PCR PROCESS AND TRACKING GENERATION ORDER OF GENERATED STRANDS

(71) Applicant: Industry-Academic Cooperation Foundation, Yonsei University, Seoul (KR)

(72) Inventors: Du Hee BANG, Seoul (KR); Hyeon Seob LIM, Seoul (KR); So Yeong JUN, Seoul (KR)

(73) Assignee: Industry-Academic Cooperation Foundation, Yonsei University, Seoul (KR)

(21) Appl. No.: 18/039,147

(22) PCT Filed: Nov. 23, 2021
(86) PCT No.: PCT/KR2021/017283

§ 371 Date: May 26, 2023

(30) Foreign Application Priority Data
Nov. 27, 2020 (KR) 10-2020-0162340

(51) Int. Cl. C12Q 1/6869; C12Q 1/6806; C12Q 1/686

(52) U.S. Cl. CPC C12Q 1/6869; C12Q 1/686; C12Q 1/6806

(57) ABSTRACT

The present invention relates to a method capable of making one cluster by connecting information of strands generated during a PCR process and tracking the generation order of the generated strands. More specifically, the present invention uses a UID-containing primer so as to enable all parent strands and daughter strands to share one UID, and uses the shared UID so as to connect two strands (parent strand and daughter strand) and furthermore extend to and connect a granddaughter strand, thereby enabling connection to all progeny strands derived from a first copied strand. Accordingly, the present invention is capable of not only making one network (cluster), but also identifying the generation order of strands generated during an amplification process, constructing lineage of amplification, and observing error patterns.

### TECHNICAL FIELD

The present invention relates to a method for generating a consensus sequence for detecting a target nucleic acid using a P2P network method.

The present invention claims the priority based on Application No. 10-2020-0162340, filed Nov. 27, 2020, entitled “METHOD CAPABLE OF MAKING ONE CLUSTER BY CONNECTING INFORMATION OF STRANDS GENERATED DURING PCR PROCESS AND TRACKING GENERATION ORDER OF GENERATED STRANDS”, and all contents in the literature of that patent application are hereby incorporated by reference in their entirety.

### STATEMENT REGARDING SEQUENCE LISTING

The Sequence Listing associated with this application has been submitted electronically in ASCII format, and is hereby incorporated by reference into the specification in its entirety. The name of the text file containing the Sequence Listing is 5142_0030001_SequenceListing_ST25. The file size is 28,523 bytes, was created on May 26, 2023, and is being submitted electronically via USPTO's patent electronic filing system.

### BACKGROUND ART

To manage cancer and provide clues for treatment, tumor mutations need to be identified. Further, early detection and continuous monitoring of tumor mutations are required because tumor mutations evolve over time and induce recurrence. Targeted rearrangement for identifying the somatic mutations of circulating tumor DNA (ctDNA) in a liquid biopsy sample is a good choice for the long-term monitoring of minimal residual disease (MRD) because the sample can be easily obtained from a blood draw and surgery or a painful needle biopsy is not required.

However, since ctDNA derived from tumor cells in the related art is generally present at very low levels in cell free DNA (cfDNA), it is difficult to confirm whether the low proportion of alleles observed was ctDNA or simply a sequencing or polymerase error. Therefore, there is a need for a method of reducing the error rate in order to accentuate the signals of tumor alleles. Recently, a method of generating a consensus sequence from a molecule tagged with an adapter containing a unique identifier (UID) by ligation has been usually used. The method using ligation in this manner allows a daughter molecule amplified from a starting molecule to be grouped using a UID sequence by connecting an adapter including a UID to the starting molecule to prepare a next generation sequencing (NGS) library for hybridization capture. Among daughter molecules including the same UID sequence, molecules including errors generally do not have a large proportion such that consensus sequence errors of daughter molecules can be removed from such a ligation-based method.

Meanwhile, to perform long-term MRD monitoring, there is a need for a quick and economical method for monitoring various personalized target mutations. However, the current technique is based on hybridization capture, which requires 2 to 3 working days and high costs. In addition, even when up to 200 genes are targeted, the current technique exhibits a ratio to target of 20-30%, and such a ratio decreases as the number of target genes decreases. Such a low target ratio makes data costs higher than expected. Therefore, the hybridization capture-based method is not the most efficient method of monitoring various personalized targets.

Therefore, there is a need for a quick and economical method capable of monitoring various personalized targets, unlike methods in the related art.

### DISCLOSURE

### Technical Problem

Therefore, an object of the present invention is to provide a method for generating a consensus sequence for detecting a target nucleic acid, the method including: amplifying DNA fragments from a sample using polymerase chain reaction (PCR) with primers containing adapter sequences, flanking sequences, and UID sequences, in the direction from the 5′ end to the 3′ end;
obtaining sequence information of the amplified DNA fragments through the PCR; and
generating a cluster using a peer-to-peer (P2P) network method based on the obtained sequence information.

Another object of the present invention is to provide a kit for generating a consensus sequence for detecting a target nucleic acid, including a PCR primer including adapter sequences, a flanking sequence and a UID sequence.

### Technical Solution

To achieve the objects described above, the present invention provides a method for generating a consensus sequence for detecting a target nucleic acid, the method including: amplifying DNA fragments from a sample using polymerase chain reaction (PCR) with primers containing adapter sequences, flanking sequences, and UID sequences, in the direction from the 5′ end to the 3′ end;
obtaining sequence information of the amplified DNA fragments through the PCR; and
generating a cluster using a peer-to-peer (P2P) network method based on the obtained sequence information.

In the following examples, model experiments were conducted using an oligonucleotide including a barcode consisting of a random base sequence in order to confirm the possibility of constructing a P2P network-based cluster. Thereafter, a unique molecular identifier (UID) sequence was added to both ends of a model oligonucleotide by the 6-cycle PCR amplification of the oligonucleotide using a polymerase. Next, the sample was converted to base sequence data by an NGS method and used for analysis. That is, it was confirmed that all UID pairs included in various daughter strands made from one oligonucleotide molecule are connected to create one cluster identifier (CID), and all molecules of the corresponding CID have UIDs with the same length.

In the present invention, the PCR primer includes adapter sequences, a flanking sequence and a UID sequence.

The adapter sequences may be 17 bp to 69 bp long or 20 bp to 50 bp long, specifically 25 bp to 40 bp long, but are not limited thereto.

Meanwhile, the method for generating a consensus sequence for detecting a target nucleic acid of the present invention may additionally trim the sequence information of the amplified DNA fragments through the PCR.

As used herein, the trimming refers to filtering out reads that have a wrong flanking sequence near a barcode sequence, 1) when a phred quality value, which is the quality of each base in a fastq file generated by NGS, is less than 30, 2) a low-quality UID sequence with fixed bases different from those designed in the example or with a minimum phred quality of UID sequences of less than 25, and 3) during the analysis of barcodes of high-GC UID sequences with a GC ratio of 0.8 or higher and synthesized oligonucleotides, in order to minimize the misidentification of the UID sequences after cutting sequence information of the amplified DNA fragments through the PCR and confirming the UID sequences in the cut primer sequence.

In the following examples, considering the relatively short average length of cfDNA at approximately 173 nt, PCR primers were designed to target approximately 100 bp regions of the desired gene to facilitate amplification. The PCR primer used in the present invention includes adapter sequences, a flanking sequence and a UID sequence in the 5′ to 3′ end direction, where the UID sequence includes the repetition of N and X in the form (N)m(X)n, N is a random base, X is a fixed base, m is a constant from 2 to 5, and n may be a constant from 1 to 2. The length of the Unique Identifier (UID) sequence is not subject to a specific limitation. However, certain issues may arise. When the length of the UID sequence is shorter than the aforementioned length, the utility may be compromised due to a reduced number of usable UID sequence cases for generating the consensus sequence. On the other hand, if the length of the UID sequence exceeds the aforementioned length, the analysis time may increase significantly, and there may be a higher likelihood of specific UID sequence-containing molecules being grouped together.

For example, in the present invention, half of the molecules newly generated in a specific cycle may be generated by inserting a new first UID, and the remaining half may be generated by inserting a new second UID. Therefore, the 2ⁿ⁻ⁱ molecules of the cluster generated by the present invention may be derived from the first copied molecule in the I-th cycle, and 2ⁿ⁻ⁱ⁻¹ molecules, which are half of the molecules in the cluster, may be generated by inserting a new first UID. Then, the other half, 2ⁿ⁻ⁱ⁻¹ molecules, may be generated by inserting a new second UID. Therefore, the maximum UID number possible per cluster is 2ⁿ⁻², meaning the time point when the cluster started with the first copied molecule in the first cycle (i=1). Further, in the PCR of the present invention, the first copied strand may be generated in each cycle, and the number of molecules per cluster may be estimated by assuming that the first copied strand is the starting molecule. Assuming that the first copied strand is generated in the i-th cycle, the number of remaining cycles is n−i.

Furthermore, the number of molecules derived from the first copied strand may be assumed to be 2ⁿ⁻ⁱ. The first copied strand with only one UID in the molecule cannot be sequenced. Therefore, the number of molecules per cluster to be sequenced is 2ⁿ⁻ⁱ⁻¹ (i=1 to n).

When the fixed base is inserted between random bases, the accuracy of PCR analysis may be improved.

Meanwhile, the method of connecting the UID sequence to the primer by the ligation method in the related art has a limitation in the number of PCR cycles to include the UID sequence in the daughter strand. For example, by the ligation method in the related art, the number of PCR cycles to include the UID sequence in the daughter strand cannot be 3 cycles or more. However, PCR for including the UID sequence in the daughter strand by inserting the UID into the PCR primer rather than the ligation method as in the present invention may include 3 to 12 or 3 to 10 cycles, and 3 to 8 cycles may be preferably performed.

As used herein, the P2P network method may refer to an algorithm method including: obtaining the sequence information of a UID pair from the sequence information of DNA fragments amplified by PCR in the present invention;
grouping a second UID including first UID sequence information and grouping a first UID including second UID sequence information among the sequence information of the obtained UID pairs; and
selecting one UID sequence from the grouping of the second UID or the grouping of the first UID, and then connecting a UID sequence pair selected from the unselected UID groups.

Further, as used herein, the cluster may refer to a group including molecules derived from the same molecule formed by the P2P network method.

Since the method for generating a consensus sequence for detecting a target nucleic acid according to the present invention uses the P2P network method, it is possible to remove errors by polymerase and sequencing errors, which may occur during PCR analysis, and as a result, it is possible to know at what amplification point an error occurred.

In addition, the method for generating a consensus sequence for detecting a target nucleic acid according to the present invention can detect mutations present in circulating tumor DNAs (ctDNAs) present in trace amounts in the blood, which are difficult to detect with existing diagnostic techniques. Therefore, it is possible to diagnose cancer with only a simple blood collection without damaging the body, and at the same time, it is also possible to diagnose the presence or absence of cancer recurrence as it is possible to detect ctDNA remaining in the blood during treatment period or after surgery.

Therefore, in the present invention, the DNA of the sample may be ctDNA. According to the present invention, even trace amounts of mutations present in ctDNA may be detected. ctDNA is only described as an advantageous example according to the present invention, but the DNA of the sample in the present invention is not limited.

Meanwhile, the present invention provides a kit for generating a consensus sequence for detecting a target nucleic acid, including a PCR primer including adapter sequences, a flanking sequence and a UID sequence.

For the adapter sequences, flanking sequence, and UID sequence included in the kit of the present invention, the content described for the method for generating a consensus sequence for detecting a target nucleic acid described above may be applied as it is or mutatis mutandis.

As used herein, next generation sequencing (NGS) refers to a base sequence analysis method, which is characterized by processing a large number (millions or more) of DNA fragments in parallel unlike the existing Sanger sequencing, and can decipher a vast amount of genomic information by breaking one genome down into numerous fragments, reading each fragment simultaneously, and then combining the data thus obtained using bioinformatic techniques.

In the present invention, the polymerase used during PCR amplification can be used without limitation as long as it is any polymerase used in the art, and may be preferably KAPA HiFi polymerase.

As used herein, the term SPIDER seq refers to a P2P network-based sensitive genotype derived from an identifier for error reduction in amplicon sequencing, and specifically, refers to a P2P network-based identifier.

In the present specification, “barcode” and “UID” can be used interchangeably, and specifically, “barcode sequence” means a wider concept sequence than “UID sequence.”

As used herein, the term “target nucleic acid” refers to any nucleotide sequence encoding a known or putative gene product. The target nucleic acid may be a gene derived from animals, plants, bacteria, viruses, fungi, and the like, or a mutated gene accompanying genetic diseases. For a target gene in the present invention, for example, a nucleic acid sequence or molecule may be single- or double-stranded, and may be DNA or RNA, which may represent the sense or antisense strand. Thus, nucleic acid sequence may be dsDNA, ssDNA, mixed ssDNA, mixed dsDNA, dsDNA made into ssDNA (for example, via melting, denaturing, helicases, and the like), A-, B- or Z-DNA, triple-stranded DNA, RNA, ssRNA, dsRNA, mixed ssRNA and dsRNA, dsRNA made into ssRNA (for example, via melting, denaturing, helicases, and the like), messenger RNA (mRNA), ribosomal RNA (rRNA), transfer RNA (tRNA), catalytic RNA, snRNA, microRNA, or PNA.

As used herein, the term “complementary binding site” or “sites where both ends bind complementarily” refers to a site capable of forming complementary base pairs between nucleotide sequences.

As used herein, the term “primer” refers to a sequence for amplifying sample fragments during PCR, and includes adapter sequences, a flanking sequence and a UID sequence in the 5′ to 3′ end direction.

As used herein, the term “detection,” “sensing” or “diagnosis” refers to confirmation of the presence or absence of a target and the presence or characteristics of a pathological state according to the presence or absence of the target.

When one part “includes” one constituent element in the present invention, unless otherwise specifically described, this does not mean that another constituent element is excluded, but means that another constituent element may be further provided.

Unless otherwise defined in the present specification, all technical and scientific terms used have the meaning typically understood by a person with ordinary skill in the art.

As used herein, singular forms include plural references unless the context clearly dictates otherwise. Furthermore, unless otherwise indicated, nucleic acids are written left to right in a 5′ to 3′ direction, and amino acid sequences are written left to right in the amino to carboxyl direction, respectively.

Hereinafter, the present invention will be described in detail through Examples. However, the following Examples are provided only for more specifically describing the present invention, and it will be obvious to a person with ordinary skill in the art to which the present invention pertains that the scope of the present invention is not limited by these Examples according to the gist of the present invention.

### Advantageous Effects

According to the present invention, sequence information obtained from a sample is used to generate a cluster using a P2P network method, thereby having an effect capable of quickly and economically removing polymerase errors and sequencing errors and recognizing when the errors occur.

The effect of the present invention is not limited to the aforementioned effects, and it should be understood to include all possible effects deduced from the configuration of the invention described in the detailed description or the claims of the present invention.

### DESCRIPTION OF DRAWINGS

FIG. 1 illustrates a schematic view of the UID system of the present invention. (A) An example of a simplified ligation-based UID system. The UID attached by ligation secures the identity of the original molecule in this system. (B) When integrating UIDs through PCR primers, UIDs are overwritten on a repeated PCR cycle. (C) Two strands are connected using a shared UID. The small red blocks of the sequence represent nucleotide variants and the small yellow blocks of the sequence represent polymerase or sequencing errors introduced in the preparation step.

FIG. 2 illustrates a model experiment showing the possibilities of FIG. 2: cluster configuration. (A) Schematic image of the experiment. Oligonucleotides were designed so as to include a 12-nt UID content for molecular identification. Primers were designed so as to have UID and adapter sequences for an Illumina sequencing platforms (B) Number of Paired-UIDs (nPairedUID). (C-D) GC content (%) of left UID (C) and right UID (D). (E) Comparison of nPairedUID between UIDs in normal-GC (<80/a) and high-GC (>=80%) groups. Group comparisons were performed with a two-tailed Wilcoxon rank sum test. (****, p value=2.50×10-152) (F) Cluster size distribution. (G) As the number of reads per UID pair and cluster, pairs and clusters are provided in the order in which the ranks are specified. (H) UID pair distribution per cluster. (I) Specificity (%) of clusters before and after the modification of the UID content within a Hamming distance of 2, where clusters are given in the rank order. (J) The redundant distribution of given cluster sizes. (K) Representative lineage of clusters in which sequencing errors were observed.

FIG. 3 illustrates the performance of a P2P network-based identifier (SPIDER-seq) for detecting single mutations (A and B) and multiple mutations (C to E). (A) Comparison of VAFs observed using SPIDER-seq with known VAFs provided by the manufacturer. The average VAF observed in repeated experiments for each sample is indicated. Pearson r=0.99871 (B) Error (%) comparison for a method such as base counts in raw bam files, base counts using UID pairs, and base counts using clusters (SPIDER-seq). Error bars indicate the standard error of the mean. A method comparison was performed with the Wilcoxon signed-rank test. (**, p value between raw bam and SPIDER-seq=3.91×10-3, p value between UID pair and SPIDER-seq=3.91×10-3) Non-reference alleles were considered errors. (C) Comparison of VAFs observed using SPIDER-seq with known VAFs provided by the manufacturer. The average VAF observed in replicate experiments for each sample and variant is indicated. Lines are linear fits. Pearson r=0.881145 (B) Error (%) comparison for a method such as base counts in raw bam files, base counts using UID pairs, and base counts using clusters (SPIDER-seq). Error bars indicate the standard error of the mean. A method comparison was performed with the Wilcoxon signed-rank test. (****, p value between raw bam and SPIDER-seq=1.75×10-7, p value between UID pair and SPIDER-seq=2.91×10-7) Non-reference alleles were considered errors. (E) Error (%) over positions. Non-reference alleles were considered errors.

FIG. 4 illustrates the results of applying the method of the present invention to a library prepared by UID ligation. (A) Schematic image of CID-based UIDs for shotgun sequencing libraries. (B) Comparison of VAFs observed using the present inventors' method with known VAFs provided by the manufacturer. The average VAF observed in replicate experiments for each sample and variant is indicated. Pearson r=0.93264 (C) Mutation identification in hybridization capture data of 1%, 0.5%, 0.25% and 0.125%. Each row corresponds to a single sample of a single replicate experiment.

FIG. 5 (FIG. S1) illustrates a schematic image for describing the process of triggering various networks in a single starting molecule.

FIG. 6 (FIG. S2) illustrates a schematic workflow for the UID connection algorithm. Paired-UIDs connected to existing UIDs were added recursively until there are no more paired-UIDs to be added. UIDs indicated in red exhibit newly added UIDs.

FIG. 7 (FIG. S3) illustrates the description for the case in which the cluster is damaged. When the UID pair is lost in the middle of the connection, the cluster splits into two parts.

FIG. 8 (FIG. S4) illustrates the concept for lineage construction.

FIG. 9 (FIG. S5) illustrates the phylogenetic tree obtained from clusters with a specificity <90%. Twenty UIDs were randomly selected to display error patterns.

FIG. 10 (FIG. S6) illustrates the error analysis results introduced at the junction. Error frequencies were low in most taxa. Errors (%) are indicated at the specified length of the node.

FIG. 11 (FIG. S7) illustrates cluster analysis results in QIAGEN Multiplex PCR polymerase (QM) and Phusion polymerase (PH) experiments.

FIG. 12 (FIG. S8) illustrates the phylogenetic tree of QM polymerase obtained from clusters with a specificity <90%. Twenty UIDs were randomly selected to display error patterns.

FIG. 13 (FIG. S9) illustrates the phylogenetic tree of PH polymerase obtained from clusters with a specificity <90%. Twenty UIDs were randomly selected to display error patterns.

FIG. 14 (FIG. S10) illustrates the phylogenetic tree of a cluster representing the non-reference genotype.

FIG. 15 (FIG. S11) illustrates the minimum data requirements for analyzing 0.125% of the mutations.

FIG. 16 (FIG. S12) illustrates the experimental analysis results using hybridization capture libraries.

FIG. 17 (FIG. S13) illustrates the phylogenetic tree of clusters exhibiting non-reference genotypes observed in hybridization capture samples (WT, replicate=1).

FIG. 18 (FIG. S14) illustrates the phylogenetic tree (WT, replicate=2) of clusters exhibiting non-reference genotypes observed in hybridization capture samples.

FIG. 19 (FIG. S15) illustrates the phylogenetic tree of clusters exhibiting non-reference genotypes observed in hybridization capture samples (WT, replicate=3).

FIG. 20 (FIG. S16) illustrates the phylogenetic tree (WT, replicate=4) of clusters exhibiting non-reference genotypes observed in hybridization capture samples.

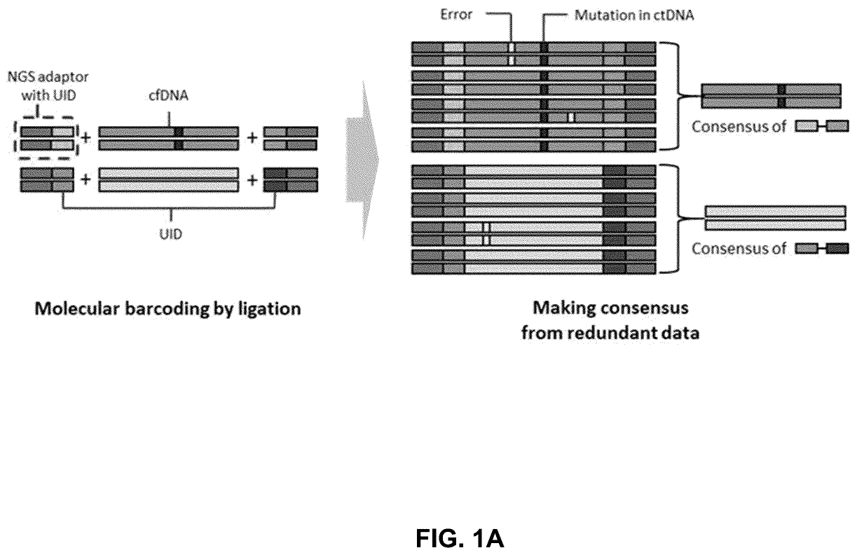

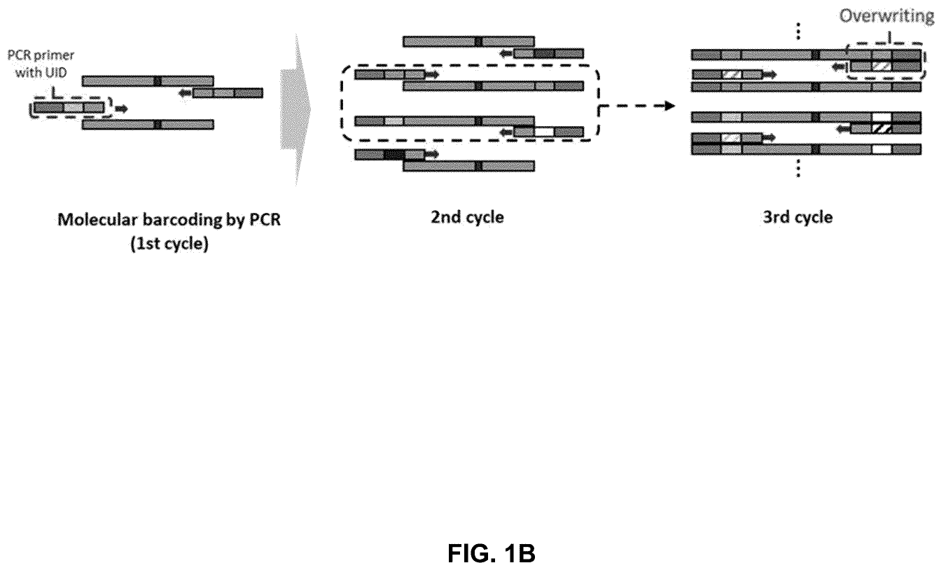

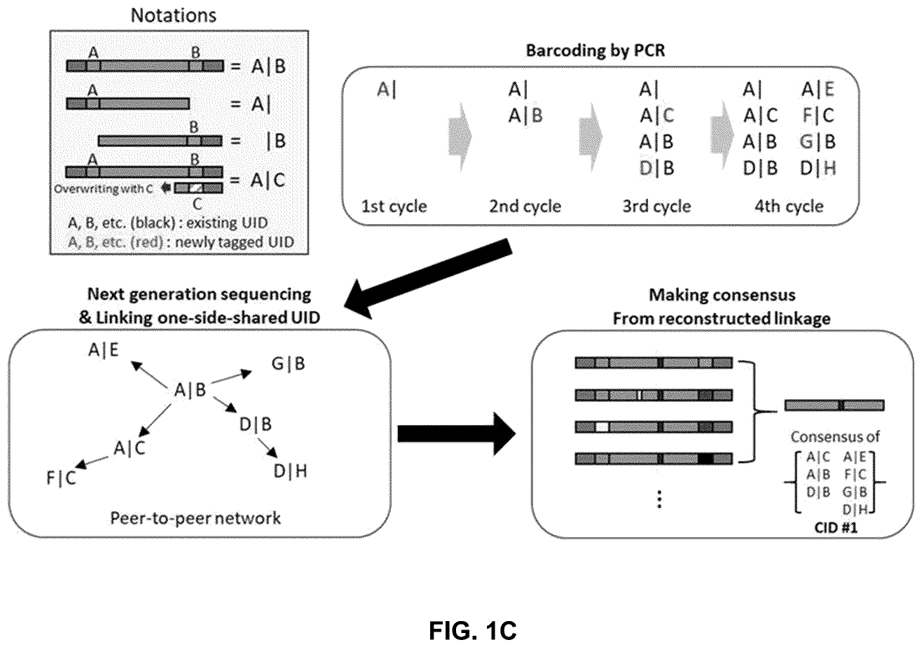

### MODES OF THE INVENTION

Hereinafter, the present invention will be described in more detail through Examples.

### Examples

1. Methods

Materials

A model experiment for demonstrating SPIDER-seq performance in the present invention was planned, and oligonucleotide sequences were designed, ordered and obtained through Integrated DNA Technologies in order to be used for the model experiment. Oligonucleotides were designed so as to mimic a genomic sequence including the BRAF p.V600E mutation, and were designed to be 173 nt in length to simulate the general length of plasma-derived cfDNA.

A portion of the genomic sequence was replaced with random base 12-nt sequences (12nt degenerate bases) to distinguish each DNA molecule (Table S8).

In the case of experiments designed to demonstrate the feasibility of SPIDER-seq for ctDNA detection, the present inventors used Seraseq™ ctDNA Mutation Mix v2 (Seracare), which is mock cfDNA in which mutated genes are mixed at a frequency of 0 to 1% (Table S9). Details on the frequency and concentration of each genetic variant were provided by the manufacturer.

PCR Primer Design

Since the average length of cfDNA is as short as 173 nt, PCR primers which target a region of about 100 bp in a target gene were designed to facilitate amplification. PCR primers are constructed as follows; a sequencing adapter, a flanking sequence and a UID sequence in the 5′ to 3′ end direction. The UID sequence (NNNNXNNNNNXNNNXNNNNNX, N=a random base and X=fixed base) consisted of 16 random bases and 4 fixed bases. The fixed bases of the flanking sequence and the UID sequence were designed so as to have different sequence combinations in order to secure sequence quality control. The sequences of all designed primers are listed in Table S8. All primers were synthesized by Integrated DNA Technologies.

Preparation of Library for Introduction and Sequencing of UID

Sequencing libraries were prepared by two rounds of PCR amplification. The first round of amplification was performed to introduce the UID sequence. For model experiments, 100 μM oligonucleotides were diluted 106-fold to limit the number of molecules, and then used as PCR templates. The recipe and cycling conditions for primary PCR are as follows.

PCR recipe using KAPA HiFi polymerase: a starting material (PCR template), 1 μl of a forward primer (10 μM), 1 μl of a reverse primer (10 μM), 4 μl of a 5×KAPA HiFi buffer, 0.6 μl of dNTPs (10 mM each), 0.4 μl of KAPA HiFi HotStart polymerase, and a final volume was made to be 20 μl using nuclease-free water.

PCR recipe using QIAGEN Multiplex PCR kit: a starting material (PCR template), 1 μl of a forward primer (10 μM), 1 μl of a reverse primer (10 μM), 10 μl of 2× QIAGEN Multiplex PCR Master Mix, and a final volume was made to be 20 μl using nuclease-free water.

PCR recipe using Phusion High-Fidelity DNA polymerase: a starting material (PCR template), 1 μl of a forward primer (10 μM), 1 μl of a reverse primer (10 μM), 4 μl of a 5× Phusion HF buffer, 0.4 μl of dNTPs (10 mM each), 0.2 μl of Phusion DNA polymerase, and a final volume was made to be 20 μl using nuclease-free water.

PCR conditions using KAPA HiFi polymerase: 6 cycles of 95° C. for 3 minutes, 98° C. for 20 seconds, 56° C. for 15 seconds, and 72° C. for 30 seconds; and 72° C. for 1 minute.

PCR conditions using QIAGEN Multiplex PCR kit: 6 cycles of 95° C. for 15 minutes, 94° C. for 30 seconds, 56° C. for 90 seconds, and 72° C. for 1 minute; and 72° C. for 10 minutes.

PCR conditions using Phusion High-Fidelity DNA polymerase: 6 cycles of 98° C. for 30 minutes, 98° C. for 10 seconds, 56° C. for 15 seconds, and 72° C. for 30 seconds; and 72° C. for 5 minutes.

In the case of experiments using mock cfDNA and targeting a single gene (BRAF), 1 μl of mock cfDNA corresponding to 3,697 to 4,788 hGE was used as a starting template (Table S10).

PCR recipe using KAPA HiFi polymerase: a starting material (PCR template), 1 μl of a forward primer (10 μM), 1 μl of a reverse primer (10 μM), 4 μl of a 5×KAPA HiFi buffer, 0.6 μl of dNTPs (10 mM each), 0.4 μl of KAPA HiFi HotStart polymerase, and a final volume was made to be 20 μl using nuclease-free water.

PCR conditions using KAPA HiFi polymerase: 8 cycles of 95° C. for 3 minutes, 98° C. for 20 seconds, 56° C. for 15 seconds, and 72° C. for 30 seconds; and 72° C. for 1 minute.

In the case of experiments using mock cfDNA and targeting various genes, 2 μl of mock cfDNA corresponding to 8,424 to 9,576 hGE was used as a starting template (Table S10).

PCR recipe using QIAGEN Multiplex PCR kit: a starting material (PCR template), 1 μl of a forward primer mixture (10 μM), 1 μl of a reverse primer mixture (10 μM), 10 μl of 2× QIAGEN Multiplex PCR Master Mix, and a final volume was made to be 20 μl using nuclease-free water.

PCR conditions using QIAGEN Multiplex PCR kit: 8 cycles of 95° C. for 15 minutes, 94° C. for 30 seconds, 56° C. for 90 seconds, and 72° C. for 1 minute; and 72° C. for 10 minutes.

After primary amplification, the product was used as it was in the next step without purification to prevent loss of product molecules. A total of 8 individual 50 μl reactions were performed using each of 2.5 μl of the product obtained from the primary amplification. The PCR recipe is as follows: 2.5 μl of the product of the primary amplification, 2.5 μl of NEBNext i5 primer (10 μM), 2.5 μl of NEBNext i7 primer (10 μM) (NEB), 5 μl of a 5×KAPA HiFi buffer, 0.75 μl of dNTPs (10 mM each), 0.5 μl of KAPA HiFi HotStart polymerase, and a final volume was made to be 50 μl using nuclease-free water.

Amplification was performed under the following conditions: 98° C. for 30 seconds, 98° C. for 10 seconds, 65° C. for 30 seconds, 72° C. for 30 seconds; and 72° C. for 5 minutes. Amplified products (about 300 bp) were purified using an MinElute Gel Extraction Kit (Qiagen) after agarose gel electrophoresis. Thereafter, the product was sequenced on Illumina NovaSeq 6000 or NextSeq 500 platforms.

Raw Data Trimming

The primer sequence was cut from the raw data, and the UID sequence was confirmed in the primer region from the cut primer sequence. To minimize the misidentification of the UID sequence, low-quality sequencing reads that satisfy the following conditions were filtered out. (i) average phred quality<30; (ii) low-quality UID base sequence with fixed bases different from the designed base sequence or a minimum phred quality of UID bases<25; (ii) high-GC UID with a GC ratio≥0.8.

While analyzing the barcode content of synthesized oligonucleotides, reads with a false flanking sequence near the barcode content were also filtered out. In experimental data analysis using mock cfDNA, trimmed data were aligned to a reference genome (hg38) using BWA-MEM (version: 0.7.15). Aligned data was converted to the BAM format and indexed using SMTOOLS (ver. 1.9). Reads with mapping quality less than 55 or mapped with soft-clipping were also filtered out. Only reads that survived this filtering were subjected to subsequent steps. Some data was downsampled using seqtk (https://github.com/lh3/seqtk) in the raw data state and then used for downstream analyses, if necessary.

Clustering by P2P Network Construction

To construct a P2P network, the UID pairs for each molecule were first organized. UID pairs sharing a primary or secondary UID were grouped together to generate connections between UID pairs. Inappropriate UIDs where the number of paired-UIDs is greater than or equal to the number of PCR cycles were removed. Starting with adding one randomly selected UID to the cluster list, elements were extended by adding the paired-UID of an existing UID. Paired-UIDs were recursively added until there were no more paired-UIDs left to add. Next, the cluster was examined to confirm whether there were more UIDs than possible (that is, 2 cycles—2) and whether there were various routes between any two UIDs (designated as a multibridge). If any one of the two cases was confirmed, the cluster was considered abnormal and discarded. Next, the UID list was designated as a CID and the read IDs supporting the CIDs were saved in a mapping file and used to designate the CID of each read from the BAM formatted data.

Analysis of Barcode Present Inside Oligonucleotide Sequence

After the peer-to-peer network (P2P network) was constructed, the trimmed fastq data was used to analyze the barcode contents. The barcode content of each read was identified based on a regular expression and collected according to the CID. When one or two sequence mismatches were observed between the main barcode and other barcodes among the barcode contents of the same cluster, the barcode content was modified to be identical to the main barcode. Then, the proportion of the main barcode in one cluster (specificity of the main barcode) was calculated.

Construction of Lineage Using Cluster Information

The main UID of a specific cluster (the UID with the most paired UIDs) was considered as a first specified UID in the PCR template (first tagged UID, that is, origin UID). Thereafter, the connected UIDs were aligned alongside the existing UID using a depth-first search. After all routes were completed, a phylogenetic tree was generated using the UID as a vertex and the relationship between connected UIDs as an edge. Phylogenetic tree data was visualized as a dendrogram using the networkD3 package (https://CRAN.R-project.org/package=networkD3). To facilitate computing, a peer-to-peer (P2P) network with a UID-to-UID structure instead of strand-to-strand was constructed. The structure reverted to the stand-to-stand-based phylogenetic tree during the visualization process.

Analysis of Mock cfDNA (cfDNA Reference Standards)

To analyze substitution mutations, reads from aligned data were parsed using the pysam module of Python, and the get_reference_sequence function of pysam was used to identify targeted bases. Then, the consensus base for each targeted position was determined for each CID. Clusters with less than 2 (<2) paired reads (that is, a total of 4 reads), a size less than 3 (<3) or a dominant base frequency less than 0.7 (<0.7) were excluded. Then, the number of consensus bases supporting each A, T, C and G was determined.

For indel analysis, mutations of interest were listed in the vcf format which may be obtained using an indel caller (for example: VarDict) or through manual scripting. To confirm whether indel mutations were present in the reads, query strings corresponding to mutant and wild-type sequences were searched for within the read sequence. Sequences consisting of 10 upstream and downstream bp were attached to wild-type or mutant sequences to generate query sequences. Then, each read was genotyped as indel or wild-type, and main genotypes per CID were determined and designated. Clusters with less than 2 paired-reads (that is, a total of 4 reads), a size less than 3, or a major genotype frequency less than 0.7 were excluded.

UID Introduction and Library Preparation for Hybridization Capture Experiments

2 μl of mock cfDNA (cfDNA reference standard) (7,394 to 9,576 hGE, Table S10) was end-repaired and A-tailed using 5XER/A-tailing Enzyme Mix (Enzymatics). Then, NEBNext Adapter for Ilumina (NEB) was connected to the DNA ends using WGS ligase (Enzymatics) and the resulting products were digested using USER enzyme (NEB).

The products were indexed with custom-designed i5 and i7 primers (Table S8). Five of the eight index bases were used for the UID and the remaining three bases were used for the sample barcode. Four index primers were designed for i5 and i7, respectively, and synthesized by Integrated DNA Technologies. Indexing was performed by PCR under the following conditions: a product to which an adapter was connected, 2.5 μl of a custom i5 primer (10 μM), 2.5 μl of a custom i7 primer (10 μM), 5 μl of a 5×KAPA HiFi buffer, 0.75 μl of dNTPs (10 mM each), 0.5 μl of KAPA HiFi HotStart polymerase, and a final volume was made to be 50 μl using nuclease-free water. PCR cycling was performed as follows: 98° C. for 30 seconds, 98° C. for 10 seconds, 65° C. for 30 seconds, 72° C. for 30 seconds; and 72° C. for 5 minutes. The product was purified using 1.2× Ampure XP beads (Beckman Coulter). Finally, hybridization capture was performed by Celemics (Korea), and then sequenced on the Ilumina NovaSeq 6000 platform.

Hybridization Capture Sample Analysis

The data was first demultiplexed using 3 bp sample barcodes in the i5 and i7 indices, and then the UID sequences were extracted from the indices. Similar to the quality trimming stage of the amplicon sequencing analysis, low-quality reads satisfying the following conditions were filtered out. (i) average phred quality<30; (ii) high-GC UID with a GC ratio≥0.8. Filtered data was mapped to hg38 using BWA-MEM. Reads with a mapping quality<55 or mapped with soft-clipping were also filtered out.

Information on paired UIDs was collected for each genomic coordinate with the same start and end positions, and clusters were constructed using such genomic coordinates. The clustering and consensus base generation process is the same as that used for amplicon library analysis, except that only reads with the same start and end positions are used to construct a cluster.

Statistical Analysis

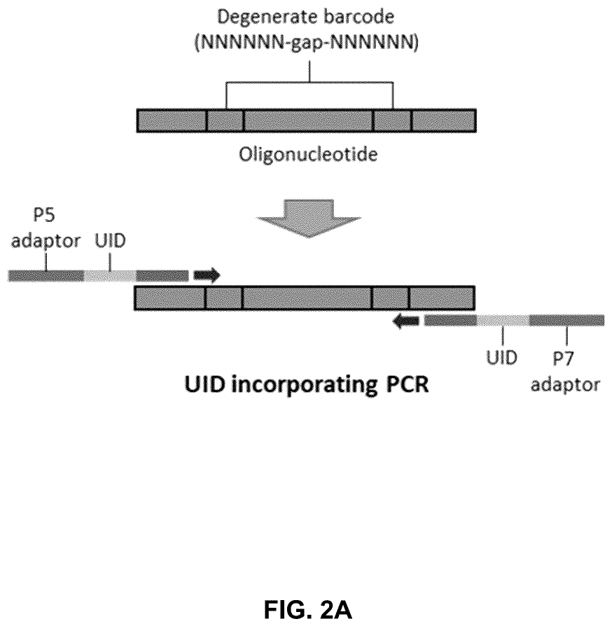

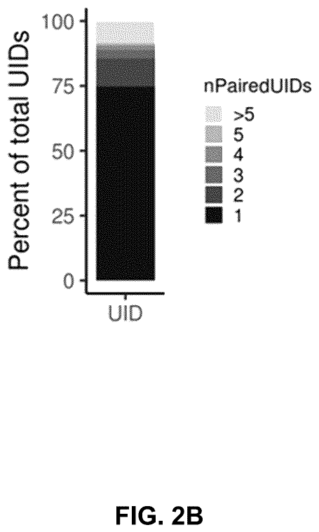

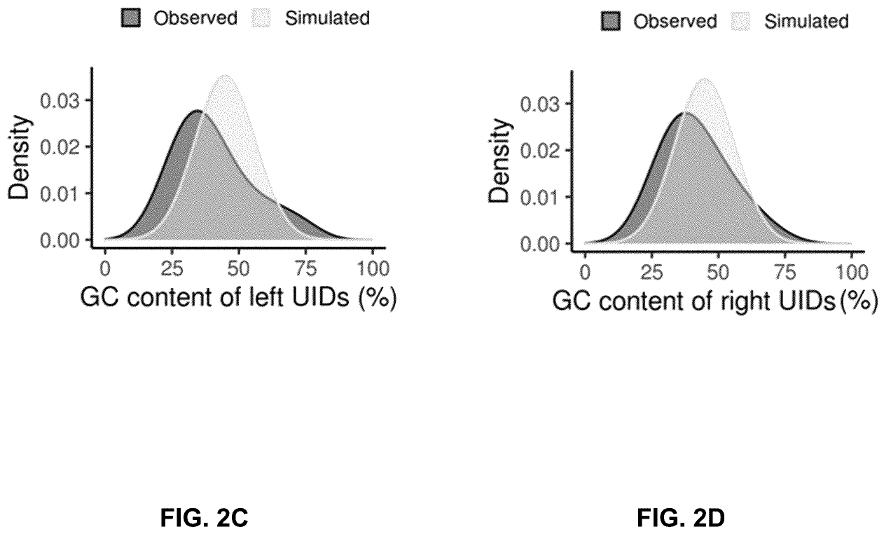

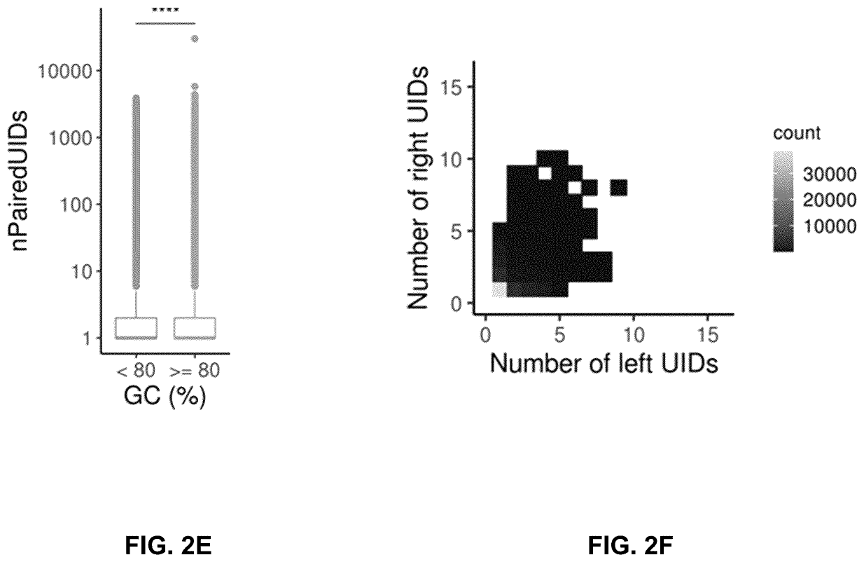

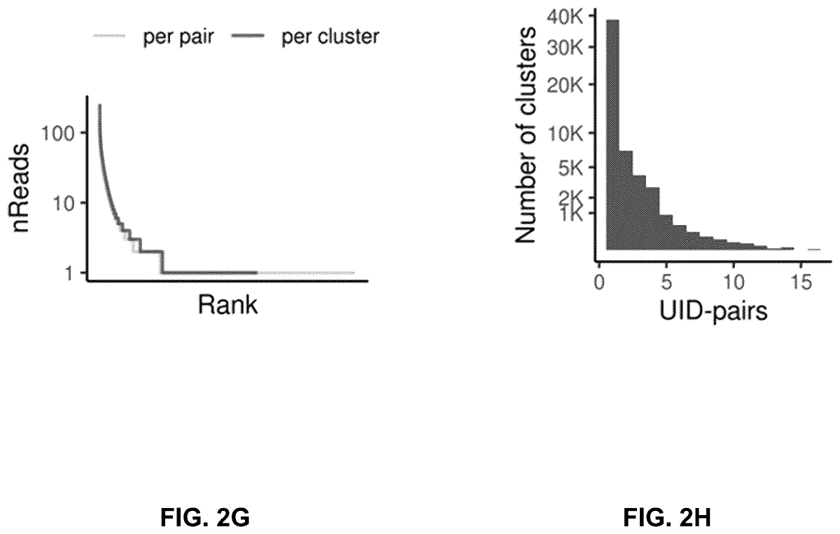

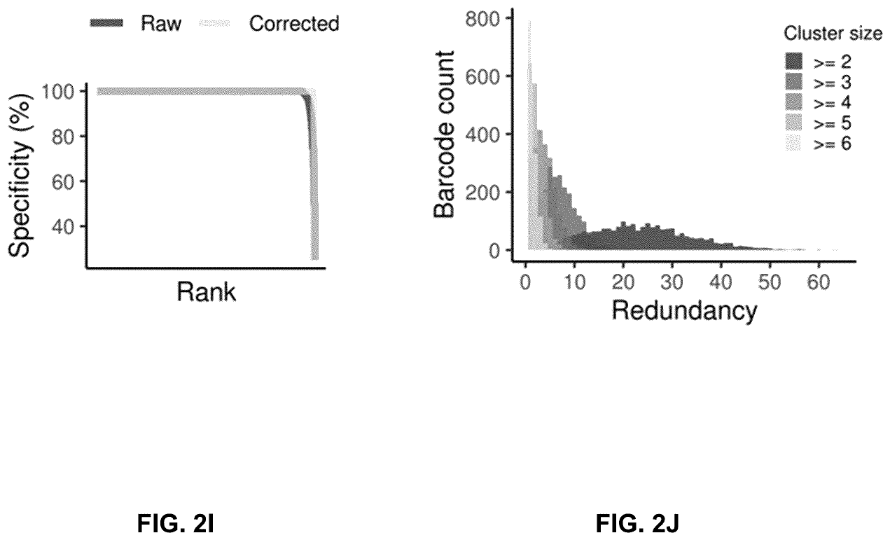

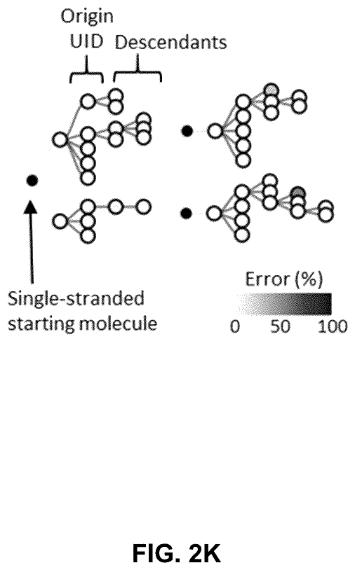

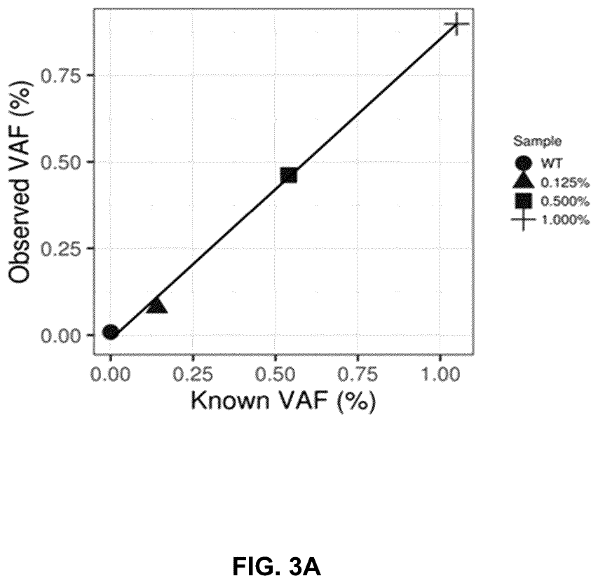

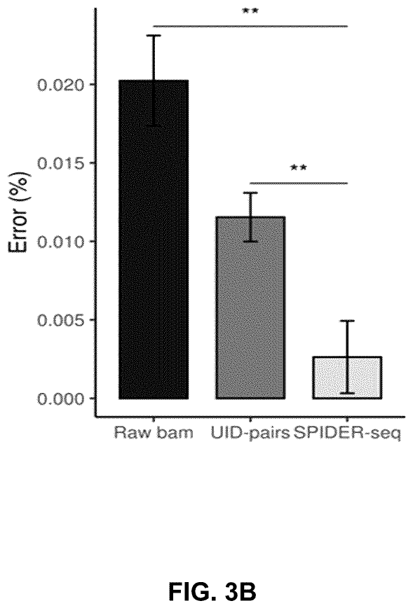

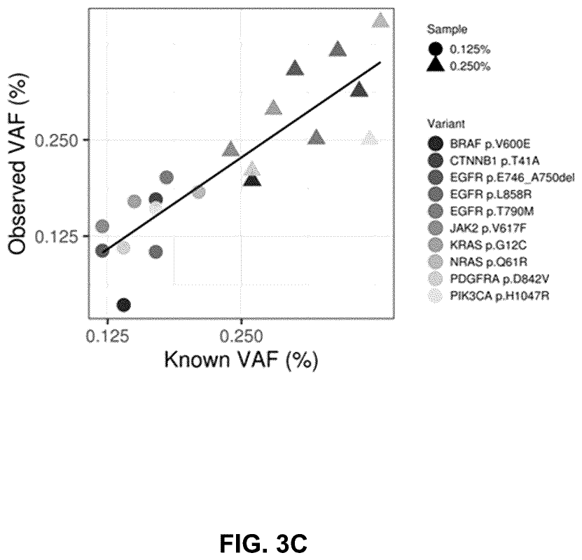

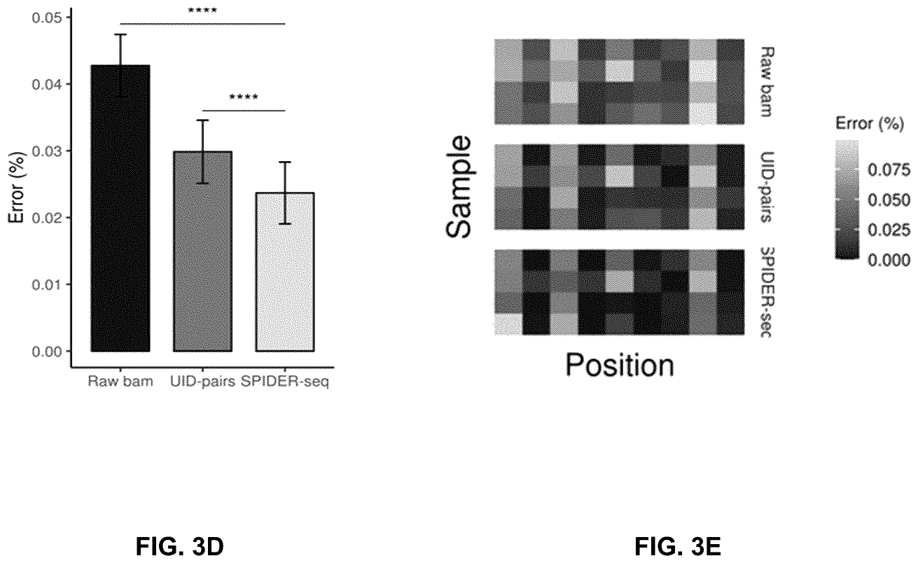

To compare differences between groups, the Wilcoxon rank sum test was used in FIG. 2E, and the Wilcoxon signed-rank test was used in FIGS. 3B, 3D and S12B.

2. Results

Possibility of Constructing P2P Network-Based Cluster

Model experiments were conducted using an oligonucleotide including a UID consisting of a 12nt random base sequence in order to confirm the possibility of constructing a P2P network-based cluster. Thereafter, a unique molecular identifier (UID) sequence was added to both ends of a model oligonucleotide by the 6-cycle PCR amplification of the oligonucleotide using KAPA HiFi polymerase (FIG. 2A). Thereafter, the samples were converted to base sequence data through a next-generation sequencing method and used for analysis. An experiment was designed so as to confirm that all UID pairs attached to various daughter strands made from one oligonucleotide molecule are connected to create one cluster identifier (CID) and all molecules of the corresponding CID actually have the same 12nt UID.

Before creating the CID, it was examined how the sequences of the UIDs could be connected. In PCR amplification, each DNA strand is repeatedly used as a template strain, and ideally, it was expected that a new UID could be attached to one parent strand per PCR cycle to create a new strand (FIG. S1). For example, it could be expected that when a parent strand with only one UID added in the 1st cycle is synthesized, daughter strands with 5 different UID pairs added from the corresponding parent strand are generated in the 2nd to 6th cycles. Similarly, newly synthesized parent strands after the second cycle can generate only four or less daughter strands because the number of subsequent remaining cycles is at most four. That is, ideally, the daughter strands can have up to 5 UID pairs in any case. As a result of base sequence analysis of this experiment, it was confirmed that in most cases, as expected, only five or less UID pairs were generated based on one UID.

Specifically, it was confirmed that most UIDs have 5 or less paired-UIDs, and only 8.41% of UIDs have 5 or more paired-UIDs (FIG. 2B). It was expected that the case of having 5 or more paired-UIDs was caused by a particularly high proportion of GC in the UID sequence. Actually, the graph of the observed GC content distribution shows a distinct right tail indicating high GC content (FIGS. 2C and 2D), and this was not observed in the ideal distribution confirmed by computer simulations that randomly generated UIDs from the UID set. In addition, it was found that parent UIDs with a GC content≥80% tended to generate more daughter UIDs (FIG. 2E). It was expected that in the case of having 5 or more paired-UIDs, a false consensus sequence could be finally created. Specifically, when a UID pair derived from normal DNA is connected to a UID pair derived from ctDNA, the base information of the mutation may be regarded as an error and removed in the process of constructing the consensus sequence. Accordingly, the present inventors set a filtering algorithm to remove UIDs with a number of paired-UIDs equal to or more than the number of cycles or cases where the GC content is 280%.

Thereafter, UID pairs having a parent-daughter relationship were found, and the UIDs in one molecule were connected one after another using the P2P network method (FIG. S2). Although connection expansion between strands was performed in a manner similar to de novo assembly, the algorithm was modified and executed such that individual UIDs were used as vertices to simplify the calculation process. Specifically, after a seed UID randomly selected to construct a connection relationship of UID pairs was selected and considered as a parent UID, all connected paired-UIDs were found, and the added paired-UIDs are considered as parent UIDs, and the method of adding new paired-UIDs was again repeated until there were no paired-UIDs to be newly added. The UID pairs thus-connected were considered as clusters, and a CID was assigned to each cluster. Through this process, 58,114 clusters made of various UID pairs were formed (FIG. 2F). For each cluster, the UIDs (the first and second sides of the amplicon, referred to as the first UID and the second UID) of each side were used in a balanced manner, and the total number of first and second UIDs per cluster (that is, the number of first UIDs+second UIDs, considered as the cluster size) was observed up to 37.

Next, it was checked how many next-generation sequencing reads per CID or UID pair could generate a consensus sequence. On average, each CID consisted of 6.283 paired reads (FIG. 2G), and a smaller number of paired reads (average 2.955) were found based on UID pairs. In terms of cluster size, clusters with a cluster size of 2 accounted for 66.05% of the total clusters, and 95,920 UID pairs were used to create clusters having a size of 3 or more, which were created by gathering various UIDs, and corresponded to 68.94% of the total UID pairs. This means that errors can be corrected using more reads when creating a consensus sequence using CIDs created by collecting various UIDs rather than using UID pairs.

Next, to evaluate the accuracy of the cluster configuration, it was checked whether the same UID was read in each CID. To observe identity, clusters consisting of only one paired-read were removed and observed. As a result, it was confirmed that most clusters included the same UID content regardless of cluster size (FIG. 2I). Even when the UIDs are not 100% identical, the sequence of UIDs is so similar that it was thought that clusters were highly likely to be created from the same UID because there was a difference in 1 and 2 bases. As a result of correcting such mismatched bases, it was found that 99.09% of the clusters had the same UID. These mismatches were expected to have occurred during PCR and sequencing.

Next, it was checked how many clusters occurred based on a UID. One starting oligonucleotide molecule in PCR may initiate a first-copied strand labeled with a different UID for each cycle (FIG. S1). Therefore, theoretically, up to 5 clusters may be generated from one oligonucleotide during 6 cycles. Even in real data, one UID was observed in various clusters in most cases (FIG. 2J). However, some barcodes were observed in 5 or more clusters, unlike the ideal case. The reason is because it was expected that due to the omission of UID pairs during the purification or sequencing stage, the connections that constituted the cluster were broken and split into various fragments (FIG. S3). This cluster splitting (FIG. 2) may be thought to be the cause for the increase in the number of clusters with a size of 2 (FIG. 2H). It was confirmed that when such clusters with a size of 2 in this way were removed, the case of generating 5 or more clusters was reduced such that the UID was not ideal. It was expected that when clusters with a size of 2 or more were selected in this manner, errors could be removed more advantageously than in the case of generating a consensus sequence based on UIDs. In addition, since the present inventors can generate various CIDs from one starting molecule, it was expected that even when information is lost during the course of the experiment, there is an advantage in that it is possible to analyze mutant bases through redundant CIDs.

Use of Lineage Reconstruction to Characterize Error-Producing Patterns

A lineage was constructed for each cluster to investigate error patterns introduced into the UID content. Parental strands with the most paired-UIDs were designated as the origin of the lineage because the earliest parental strand for each cluster was most likely to generate the most daughter strands during the entire PCR cycle. Then, by listing the connected UIDs in order, a route with a form similar to a phylogenetic tree was completed (FIG. S4). Then, it was first investigated whether errors are conserved across generations. Error patterns were examined on the basis of 1 or 2 mismatches introduced into the barcode (observed in clusters showing a specificity less than 90% before error correction). 23 barcodes were randomly selected from all barcode contents in which errors were observed to confirm when the error was introduced and whether the error continued (FIG. 2K, FIG. S5).

First, it was confirmed whether the morphology of the phylogenetic tree was normal. Theoretically, as generations increase, the number of daughter strands which can be produced decreases, so that the number of branches toward the progeny side should gradually decrease, and it was confirmed that the phylogenetic tree observed in the experiment also had a similar morphology. Overall, the number of branches was lower than the theoretical number in phylogenetic trees, which was expected to be due to imperfect amplification and loss of molecules occurring during the purification process.

Next, the pattern of errors was observed. The present inventors hypothesized that errors could be introduced in three steps. (i) 6 cycles of the amplification reaction for assigning a UID (that is, a polymerase error) (ii) secondary amplification for attaching a sequencing adapter (that is, a polymerase error), and (iii) during sequencing (that is, a sequencing error). The present inventors hypothesized that errors introduced in the first step would be conserved across generations with high-frequency, whereas errors introduced in the second and third steps would produce a low proportion of sporadic error patterns.

Experimentally, the error frequency of the individual junctions is low (FIG. S6A) and few errors are conserved across generations (FIG. S6B). This indicates that most of the observed errors were introduced during secondary amplification or sequencing (that is, steps (ii) and (iii)). Such a result was expected to be obtained because high-fidelity (HiFi) polymerase hardly generated polymerase errors during 6 cycles. Specifically, a total of 2,788 oligonucleotide molecules generated 88,982 daughter strands, and 1067,784 bases were analyzed in consideration of the 12nt barcode sequence (that is, 12 bases of barcode content per strand)×the number of daughter strands). However, the error rate reported by the manufacturer of the polymerase used in this experiment is at the level of one per 3.6×10⁶ bases, and it is reasonable that there is no polymerase error.

A similar pattern was observed even in experiments using other polymerases. The same experiment was performed using QIAGEN Multiplex PCR polymerase (hereinafter “QM”), which is known to have a higher error rate than KAPA polymerase, and Phusion polymerase (designated as “PH”), which has an error rate similar to that of KAPA polymerase. As a result, a total of 3,488 molecules generated using 138,857 daughter strands were analyzed in the QM experimental group, and 2,500 molecules generated using 96,023 daughter strands were analyzed in the PH experimental group (FIG. S7). It was confirmed that both polymerases had the same barcode for each cluster, similar to the KAPA polymerase, and it was confirmed that the correction of one or two errors introduced into the barcode content increased the identity of the barcodes in the cluster. Polymerase errors rarely occurred even when QM with a higher error rate than KAPA and PH was used, and errors were not conserved across generations (FIGS. S8 and S9).

Finally, in oligonucleotide experiments, 50,000 to 90,000 consensus sequences after error correction in thousands of initial molecules could be obtained (Table S1), in other words, this means, when starting with a sample of thousands of haploid genome equivalents (hGEs), dozens of clusters can be generated and used in the amplification process even with one or two ctDNA molecules.

Mutation Detection with Allele Frequency of 0.125%

To actually confirm whether SPIDER-seq could be used for ctDNA detection, a test was performed by obtaining mock cfDNA samples in which a variant allele frequency was adjusted to 1, 0.5, 0.125 and 0% (that is, a control). Among these, UID primers for amplifying the BRAF gene harboring the p.V600E mutation were prepared, and the vicinity of the BRAF V600 sequence was amplified using an 8-cycle PCR reaction. Using 12.2 to 15.8 ng (equivalent to 3,697 to 4,788 hGE) of mock cfDNA, an average of 215,551 strands were obtained, and an average of 113,234 clusters were generated by P2P network construction. Then, an average of 42,795 consensus sequences made from 2 or more UIDs in the clusters were analyzed. As a result of P.V600E mutation assay, mutations were successfully detected even at a variant allele frequency of 0.125%, and almost no other unintended base changes were observed (FIG. 3A, Table S2). To compare performance, analysis was also conducted with consensus sequences using UID pairs (FIG. 3B), and it was confirmed that UID pair-based consensus sequences had a higher error rate than cluster-based cases (P=3.91×10-3, Wilcoxon signed-rank test).

In the mock cfDNA sample with a variant allele frequency of 0.125%, tens to hundreds of consensus reads were confirmed to exhibit the p.V600E mutation (Table S2), meaning that many clusters were formed compared to the actual number of molecules, as described for the model nucleotide. Actually, it is expected that there will be no more than 10,000 total initial strands for amplification (that is, 2 strands×5,000 hGE), and the ideal number of mutated strands should be about 12. Therefore, this data shows that duplicate clusters using the SPIDER-seq method can compensate for possible losses during a next-generation sequencing library preparation process.

Next, the error which occurred at the p.V600E position was investigated. In addition to the p.V600E mutation (corresponding to the mutation from A to T on the genome), a mutation from A to G and a mutation from A to T were rarely observed in the mock cfDNA sample with a variant allele frequency of 1% (Table S2). As a result of reconstructing the lineage for such clusters, it was confirmed that the errors were preserved for a long time on the phylogenetic tree. This means that errors were generated by a polymerase (FIG. S10), and in particular, it was expected to be due to errors introduced during the 8-cycle amplification reaction. It was expected that the reason why more polymerase errors occurred compared to the oligonucleotide model experimental conditions was that more daughter strands were sequences because two more cycles were added in the purification step, thus increasing the possibility of errors. Similarly, errors with a high frequency were observed even at the peripheral positions of the mutation (Table S3). In this manner, since SPIDER seq can be used to connect molecules formed during the amplification process in the form of a phylogenetic tree, it could be seen that it is possible to analyze in what process errors occurred, making more accurate analysis possible.

Next, to investigate the minimum amount of data required for low-content ctDNA mutation analysis, analysis was performed by down sampling the sequencing data to 10,000 to 10,000,000 read depths. As a result, the present inventors found that 100,000 depth data is sufficient to detect mutations at a variant allele frequency of 0.125% (FIG. S11A). These results suggest that mutations can be identified in a shorter time using the MiniSeq Rapid Kit capable of generating 2 Gb of data within 5 hours. Therefore, the SPIDER-seq method was expected to be useful when examining a small number of individual samples at irregular intervals, such as monitoring of minimal residual disease. However, it was expected that 100,000 or more NGS reads would be required to generate the correct consensus sequence using more daughter strands (FIG. S11B).

Mutation Multiple Detection of 10 Genes

Next, the present inventors tested whether the SPIDER-seq method could be extended to simultaneously examine mutations at various positions. A multiplex PCR method using QM polymerase was used as an experimental method that enables simultaneous examination. As target genes, a total of 9 substitution mutants and 1 indel mutant (EGFR p.E746_A750del) were selected from among the mutants included in mock cfDNA (Table S4), and next-generation sequencing library preparation and mutation analysis were performed from mock cfDNA whose average variant allele frequency was adjusted to 0.25, 0.125 or 0%. As a result, it was confirmed that the mutant allele frequencies of the tested substitution mutations coincided well with the mutant allele frequencies of the mock cfDNA provided by the manufacturer. It was confirmed that the average error rate was around 0.02369%, which was higher than that when one BRAF p.V600E position was previously examined with KAPA polymerase (error rate of 0.002628%) (FIGS. 3B to 3E), which was still a low value. From such a difference, it was expected that the QM polymerase introduced more errors than the KP polymerase during 8 amplification cycles.

To investigate indel mutations, the present inventors developed and used algorithms different from those used for substitution mutation analysis. Substitution mutations could be examined by counting A, T, C, and G bases at a given gene locus, whereas depending on the size of the indel mutation, countless patterns of indel mutations had to be considered. Therefore, the present inventors analyzed indels by devising the following three-step strategy. (i) Generation of variant call format (vcf) files or manual generation of target indel vcf files after analyzing indels using third-party indel analysis software such as VarDict from raw data prior to cluster generation. (ii) Generation of clusters by P2P networking. (iii) Evaluation of whether or not indel mutations stored in vcf are observed in NGS reads for each cluster. As a result of analysis of deletion mutations present in the EGFR gene based on such a strategy, actually, it was confirmed that in some clusters, deletions were observed in most reads within the cluster (FIG. 3C). This means that it can be confirmed that indel mutations can be accurately identified by the SPIDER-seq method.

Use of Alternative Libraries for Hybridization Capture

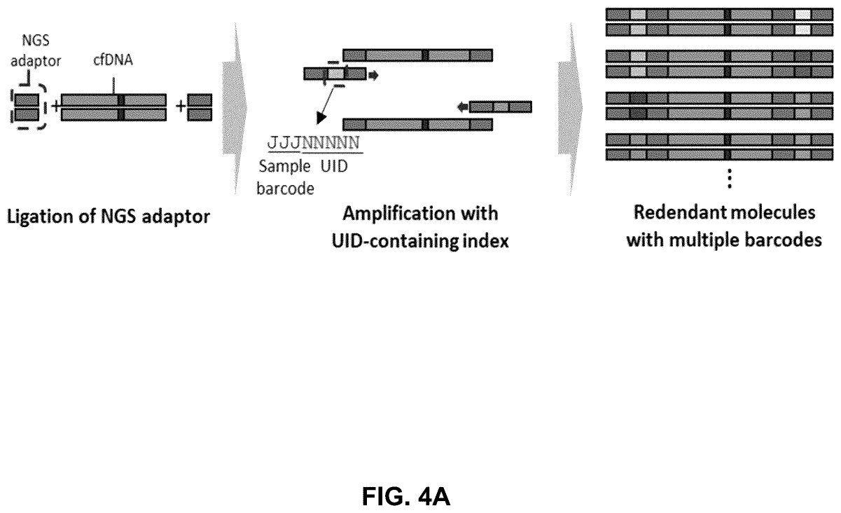

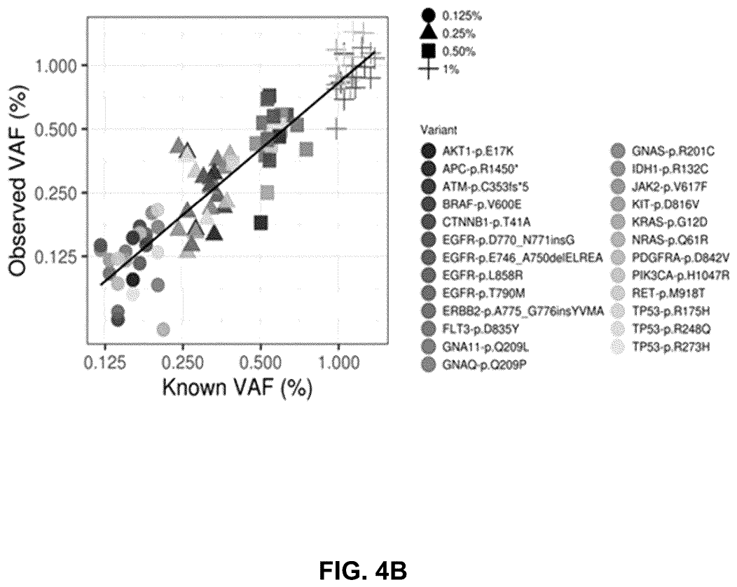

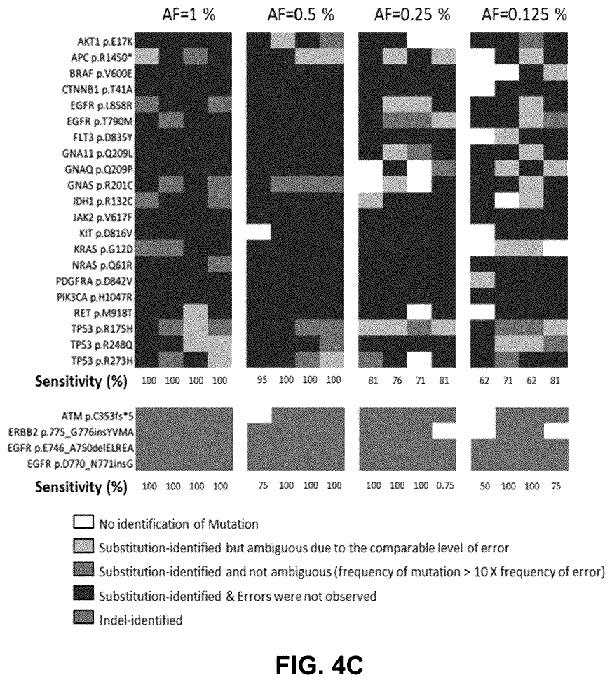

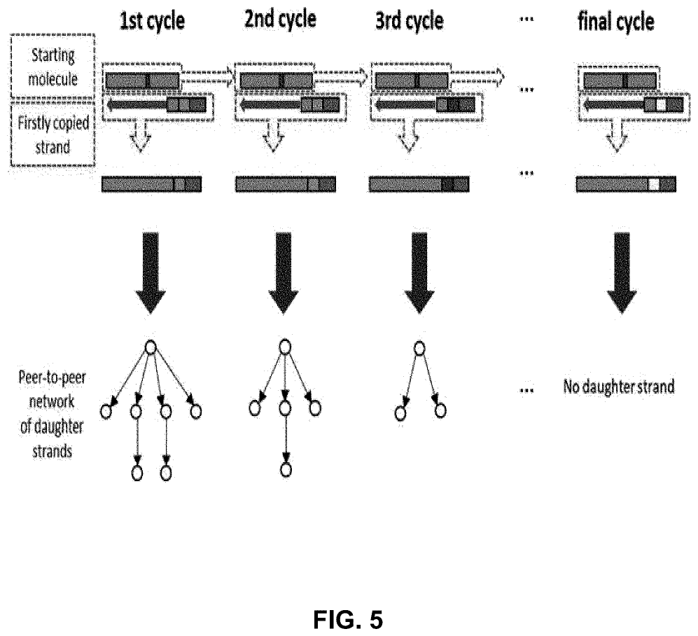

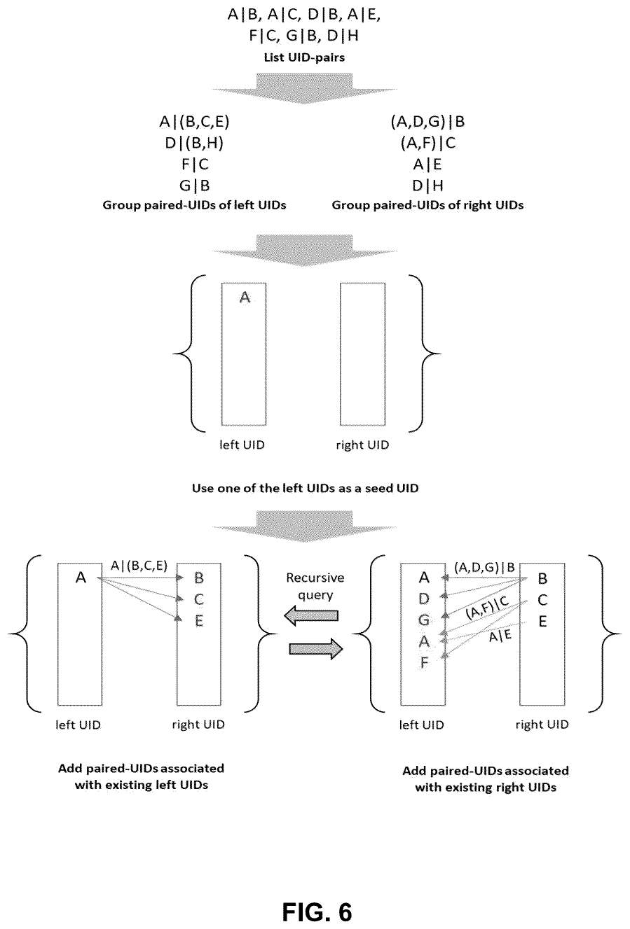

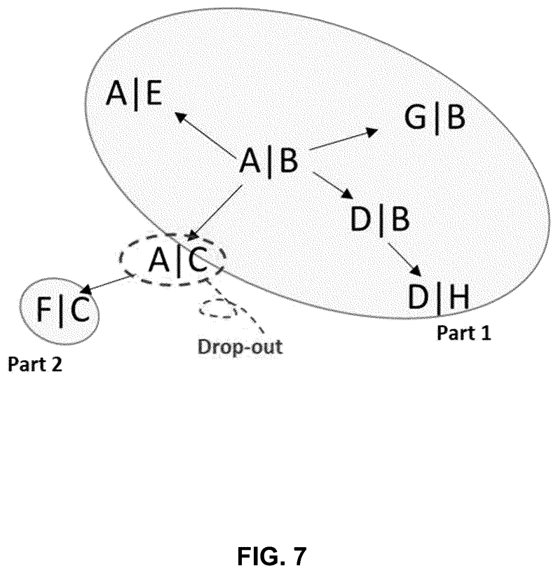

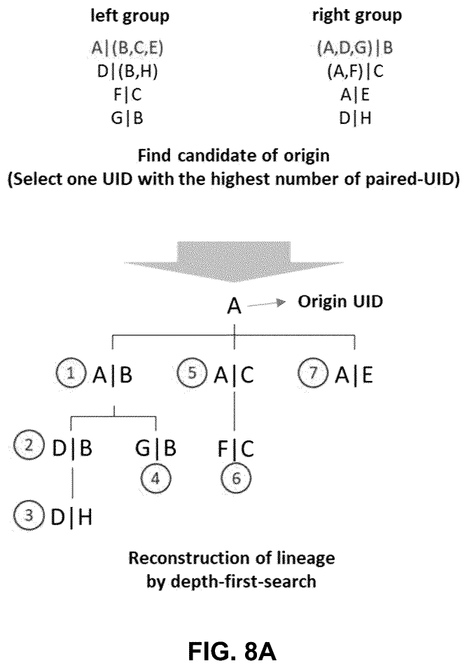

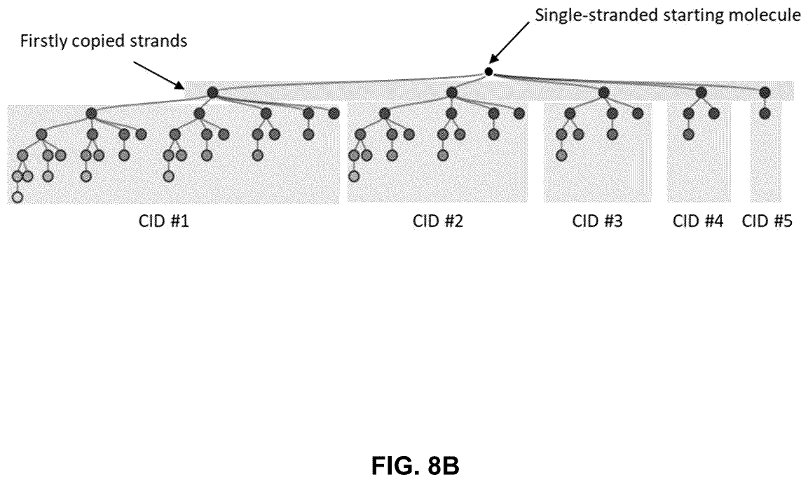

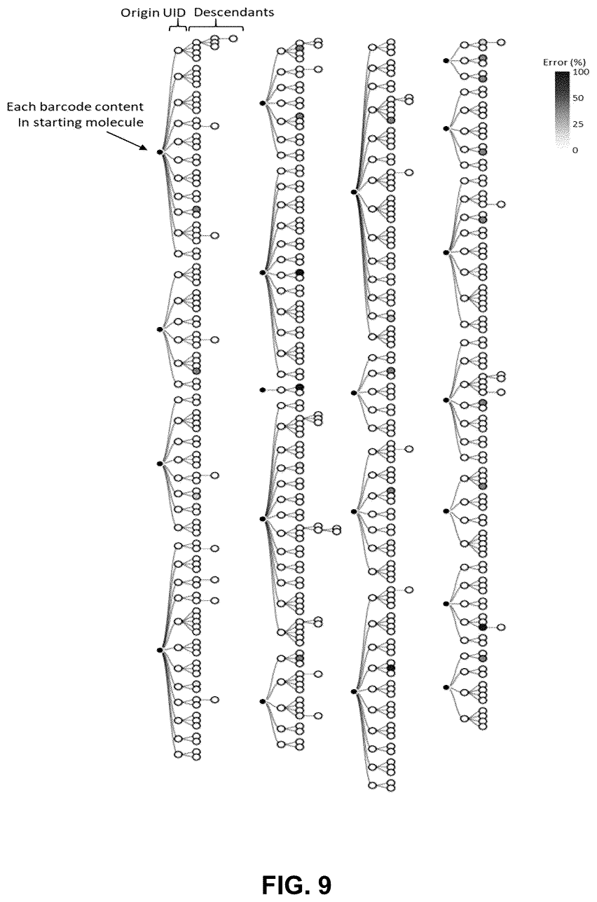

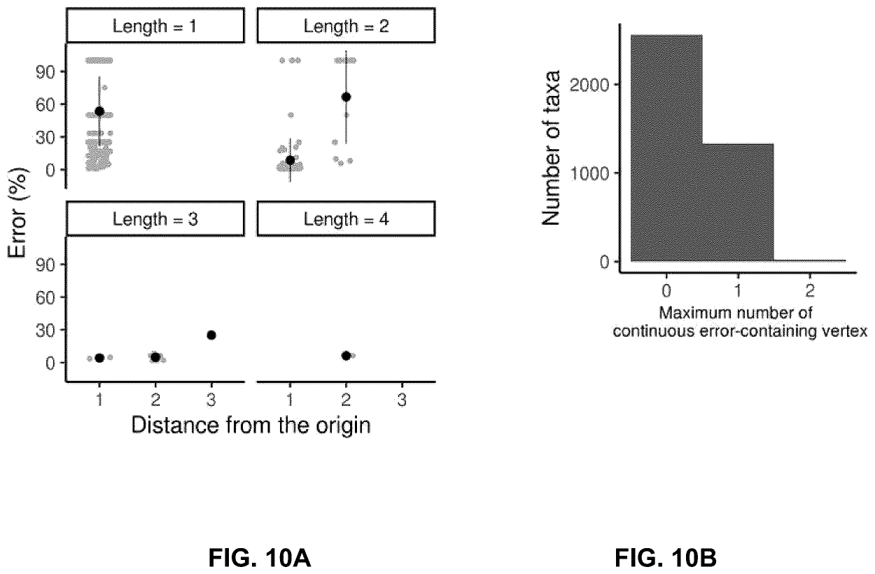

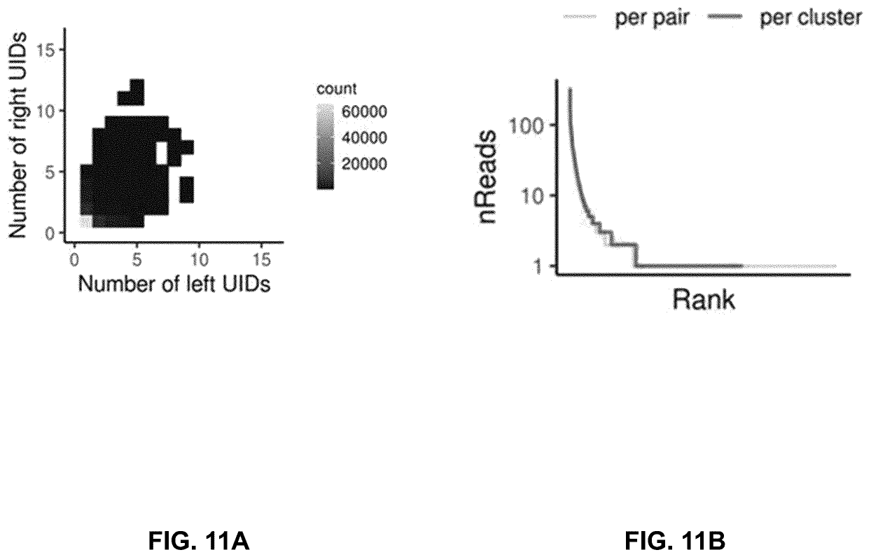

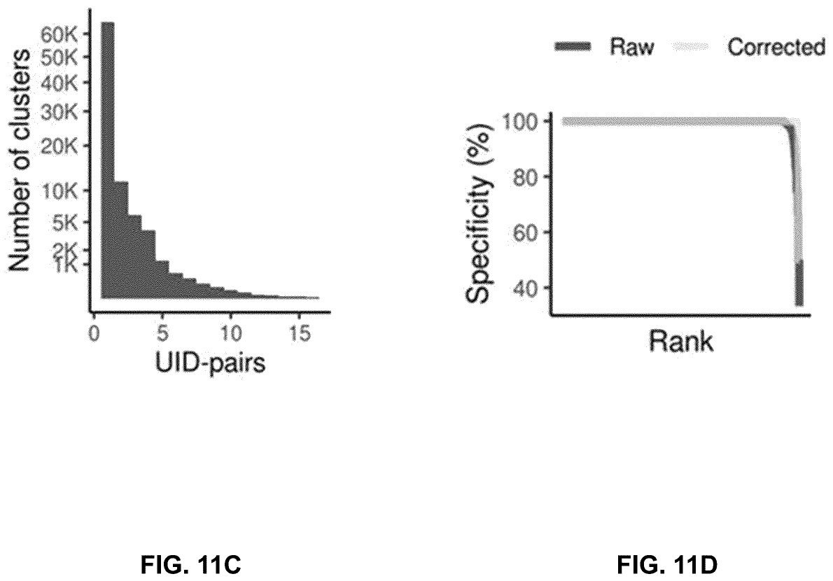

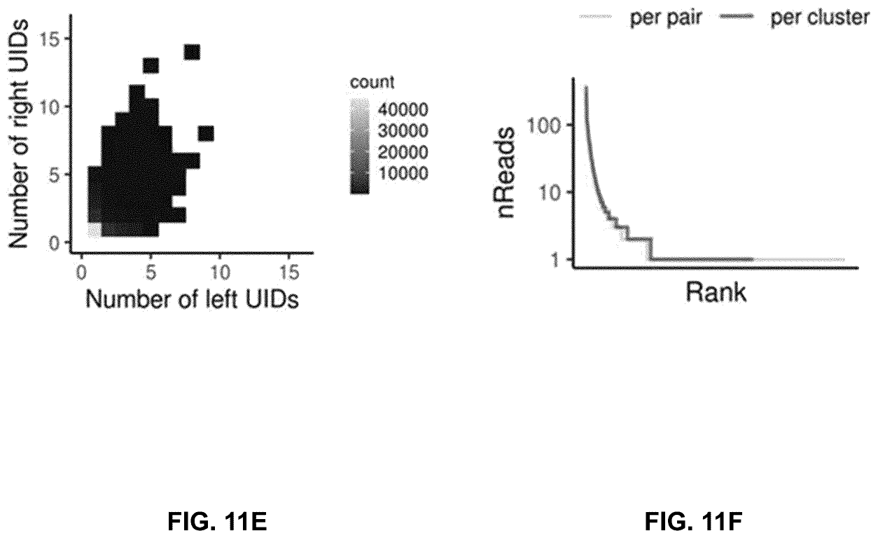

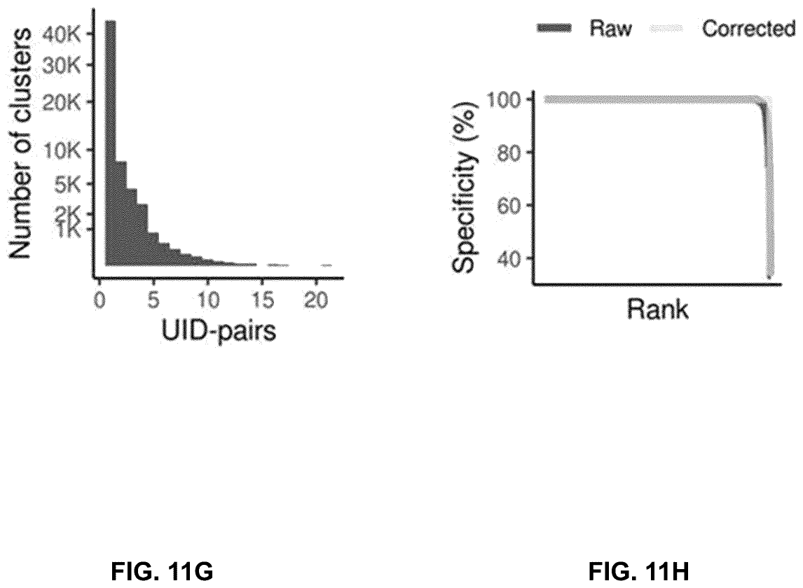

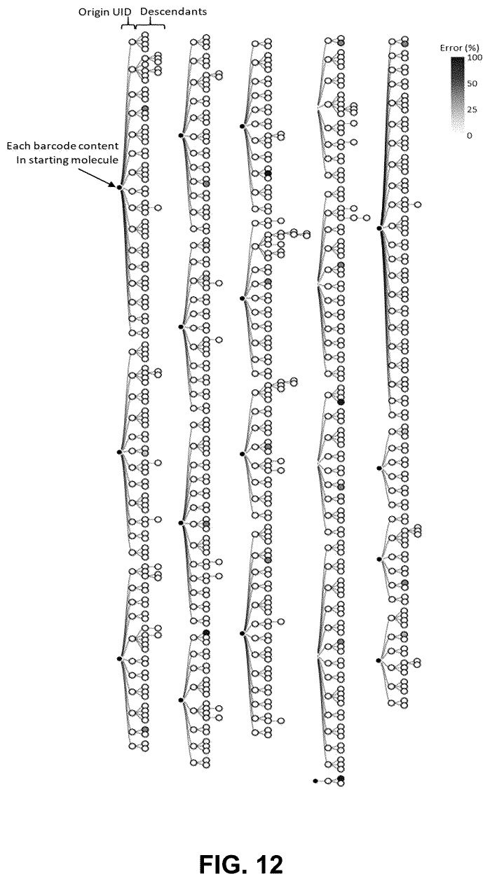

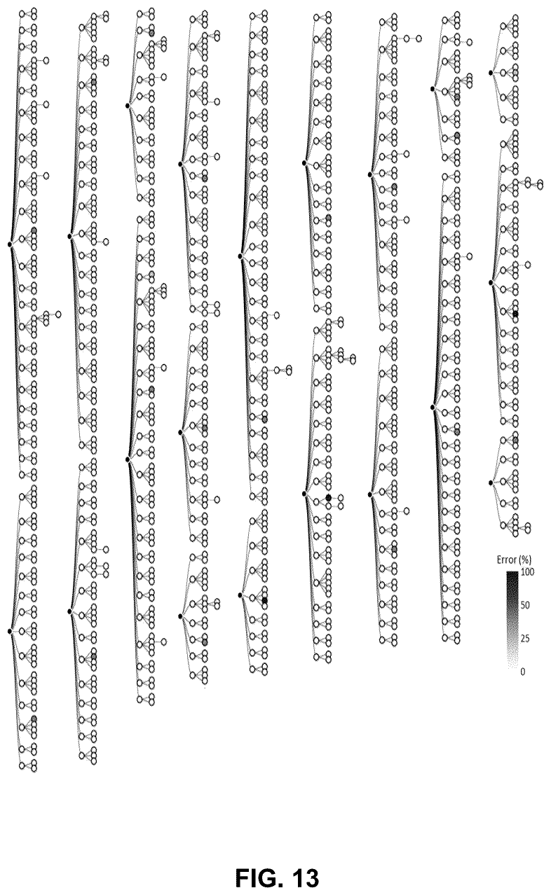

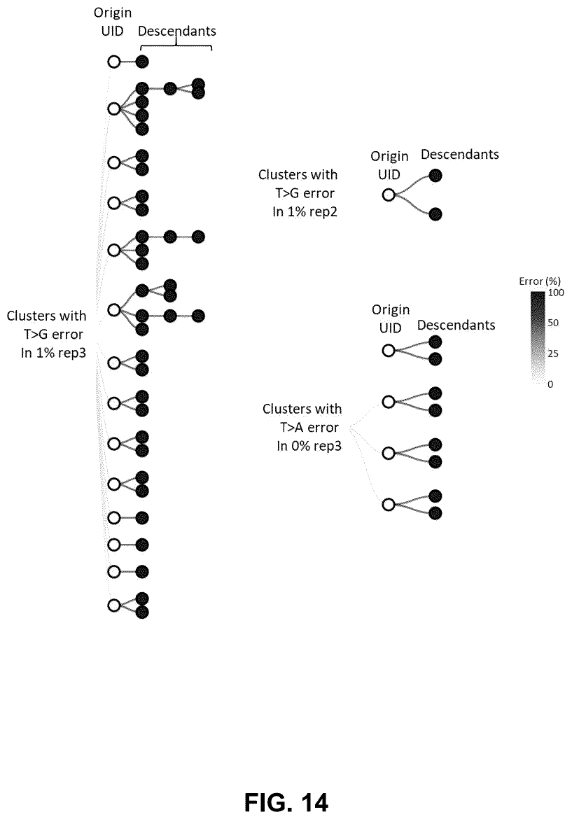

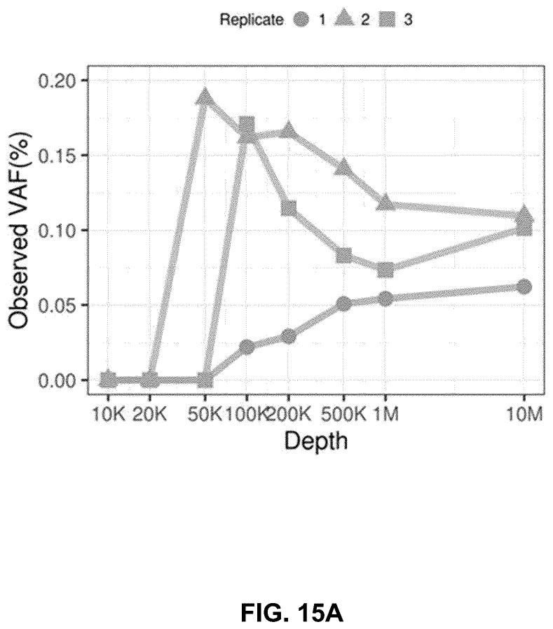

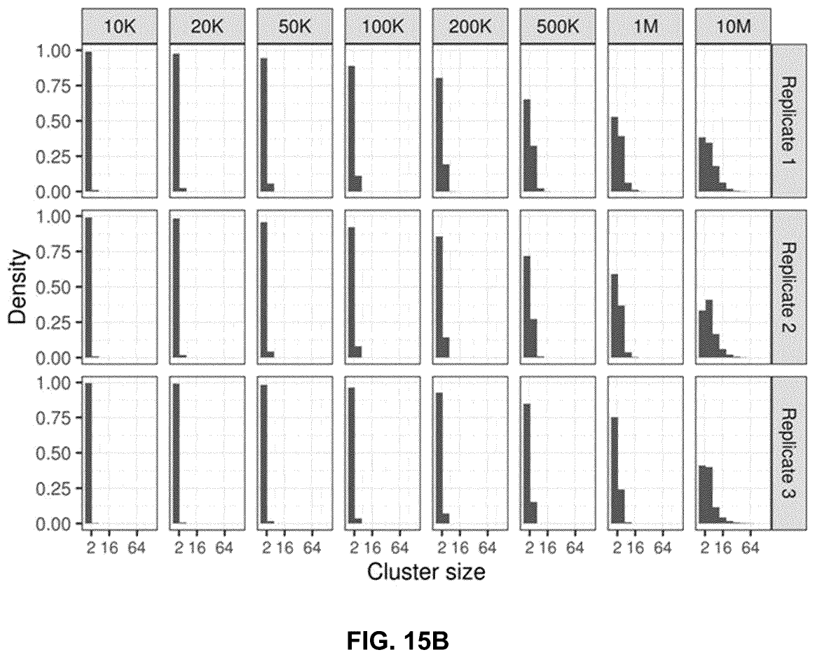

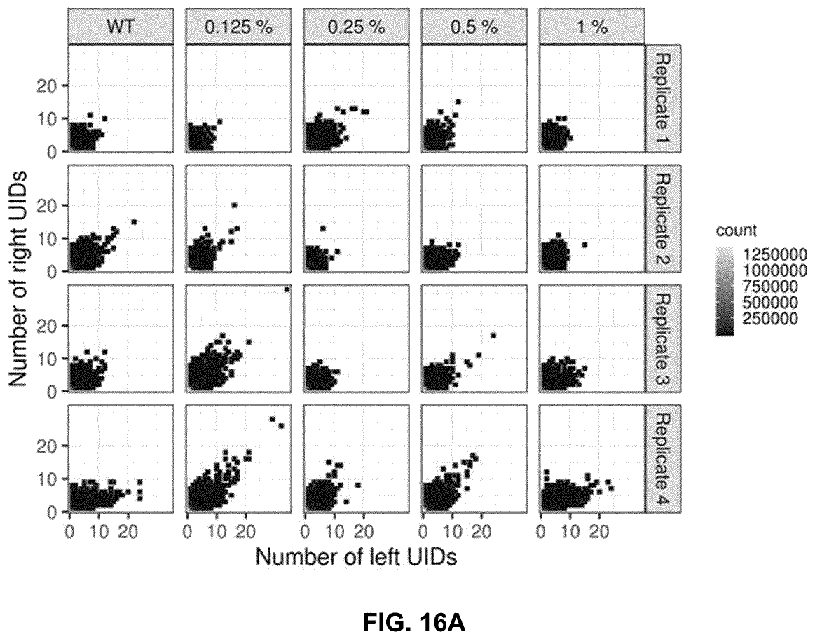

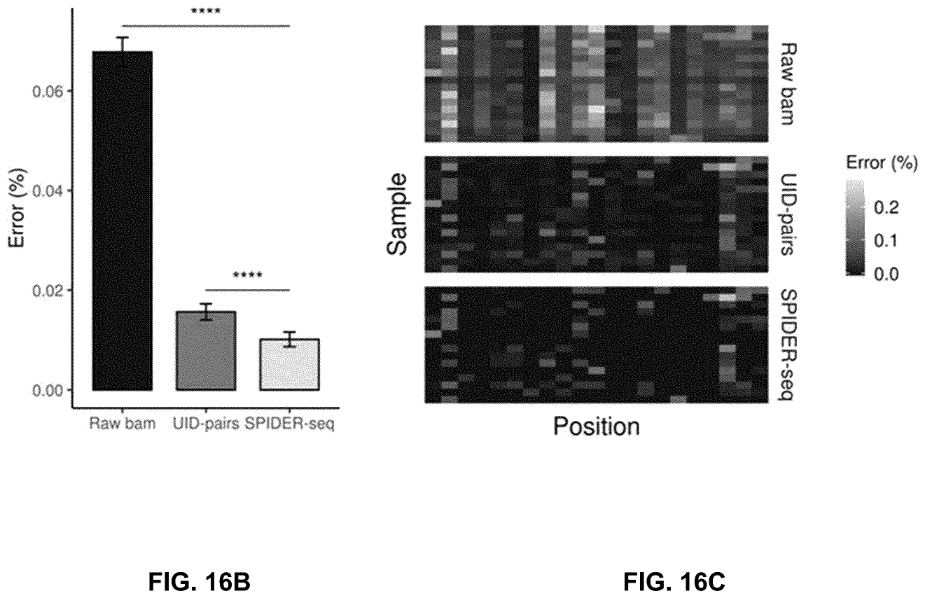

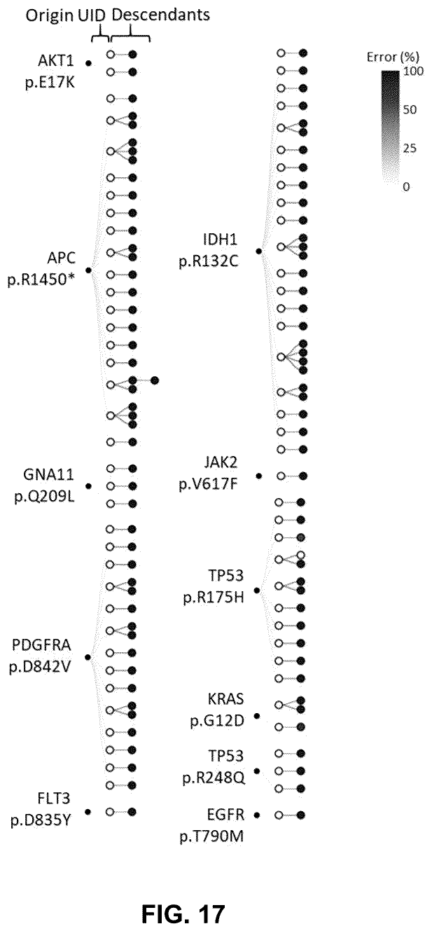

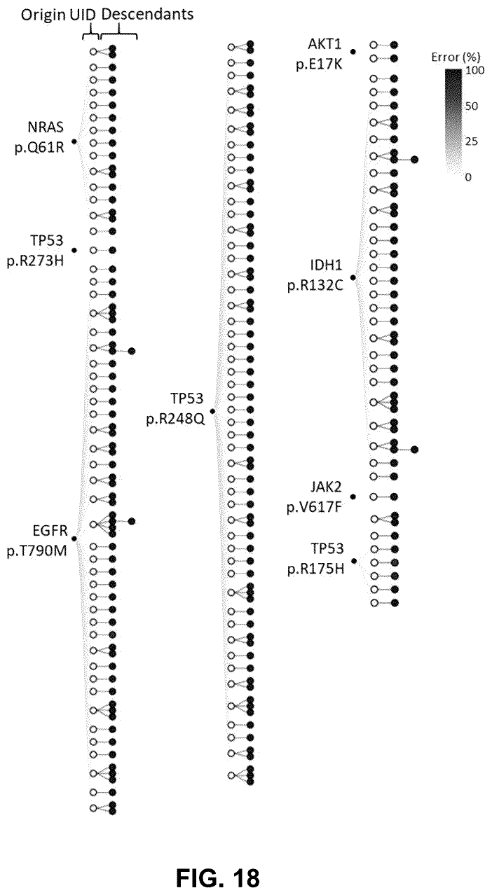

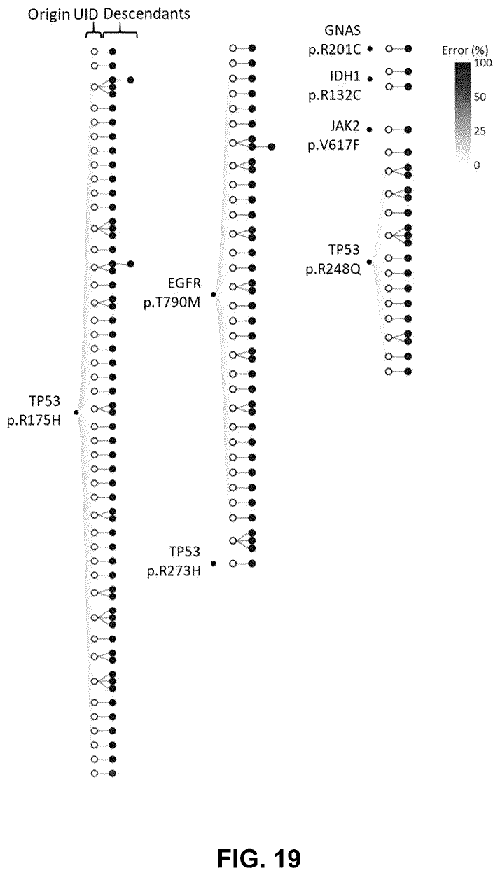

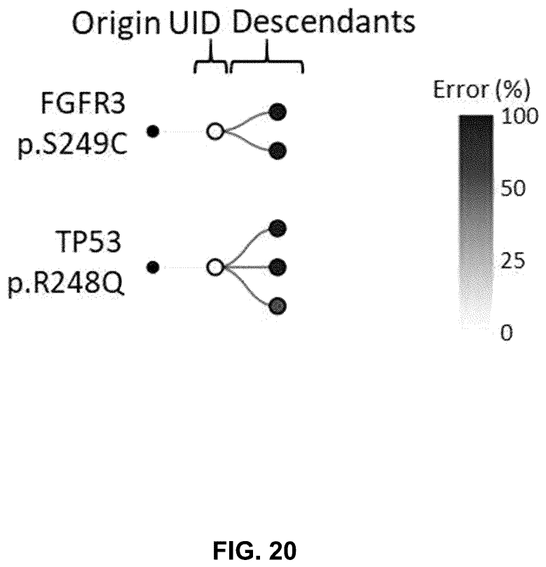

The SPIDER-seq method is originally based on an amplicon sequencing protocol, and although the goal of reducing sequencing errors by targeting a small number of positions is important, it was thought that a phylogenetic tree could also be constructed simply to track error patterns. Accordingly, the present inventors also applied the SPIDER-seq method to the library prepared based on the adapter ligation protocol. Then, the present inventors investigated where the most error-prone steps were during the preparation of target sequence libraries by the hybridization capture method. For this purpose, first, in order to assign a UID to each molecule during the process of preparing a shotgun sequencing library for hybridization capture, an experimental method was modified so as to use three bases for sample discrimination in a sequence part with a length of 8 bp, which corresponds to the index sequence of next-generation sequencing, and 5 random bases for use as a UID sequence. Then, primers including these sequences were used to amplify an adapter-linked product, and these eight bases were allowed to be read as “index read” during the sequencing step (FIG. 4A). Although it was expected that it would be difficult to label a large amount of DNA because the length of the UID was short compared to the amplicon method, the information on the location of the genomic fragments could be used as a secondary identifier because a shotgun sequencing library which randomly fragments the genome was used, so that it was able to compensate for the low diversity of the five-base UID.

To test whether a P2P network can be constructed from the shotgun DNA library, libraries were prepared from mock cfDNA engineered so as to have a genetic mutation at a ratio of 0, 0.125, 0.25, 0.5 or 1%. In this case, 8 cycles of PCR were used to introduce the UID into the PCR template. Then, hybridization capture was performed and sequencing was performed using a panel targeting 68 genes including 24 substitution mutations and 4 non-homopolymer mutations present in the mock cfDNA (Table S5). As a result of sequencing, the present inventors obtained a depth of 338,919× on average. Regions having a 100,000× depth or more, which is the minimum depth for detecting mutations present at a low rate of 0.125%, were obtained, and were regions corresponding to 21 substitution mutations and 4 non-homopolymer indel mutations (Table S6). Only regions covering 21 substitution mutations and 4 non-homopolymeric indel mutations were targeted to construct the P2P network.

Only UIDs having the same genomic coordinates were used to construct the P2P network. On average, 24,491 clusters were observed at 25 locations (Table S7), and the size of clusters was variously observed (FIG. S12A). Variant allele frequencies for substitution and indel mutations obtained based on the consensus sequence obtained from the cluster showed high coincidence with the frequencies provided by the manufacturer (FIG. 4B). Further, it could be confirmed that the error rate was reduced 6.004-fold using the CID-based consensus sequence. This result showed that SPIDER-seq can also be applied to the adapter ligation protocol. However, performance tended to be slightly reduced compared to the amplicon sequencing protocol. First, the sensitivity was not 100% in the samples with a frequency of 0.5, 0.25 and 0.125% (FIG. 4C). It was expected that such a decrease in sensitivity was probably caused by the loss of molecules during the additional experimental step of hybridization capture compared to the amplicon sequencing protocol. Second, although KAPA polymerase was used in both experiments, the basic error level observed in data which did not generate a consensus sequence (that is, 0.0685%) was higher than that in the BRAF gene locus amplification experiment (0.0202%) (FIGS. S12B, S12C and 3B). The present inventors surmised that more starting material and more sequencing data would be required to improve sensitivity. Otherwise, it was expected that more stringent filtering criteria would be required to eliminate false positive results compared to the amplicon sequencing protocol. Nevertheless, it could be confirmed that the error rate of SPIDER-seq was remarkably lower than that of raw data analysis.

The present inventors hypothesized that errors could be introduced during four stages. (i) Errors introduced during the pre-capture library preparation (that is, polymerase errors) step. In this case, errors will be conserved with high frequency in descendant molecules. (ii) Errors introduced by oxidative damage which occurs during the capture process. Errors introduced at this stage can be observed at a high frequency at specific nodes, but will not be conserved in descendant molecules. (iii) After capture (that is, polymerase errors). (iv) During sequencing (that is, sequencing errors). Errors introduced via stages (iii) or (iv) are sporadic and will be observed at low frequency. To visualize such error patterns, a phylogenetic tree of clusters showing non-reference genotypes was reconstructed (FIGS. S13 to S16). Most of the errors were found to be preserved over all quarters, implying that they were errors that occurred in stage (i). However, since clusters representing most non-reference genotypes consisted of two daughter strands, it was difficult to define the most error-prone step. The present inventors hypothesized that when clusters including errors in the case of (ii) are split into smaller clusters by the experimental loss of molecules, similar patterns can be generated.

In summary, this data indicates that the SPIDER-seq method developed by the present inventors is also applicable to the adapter ligation protocol and has a sensitivity sufficient to detect genetic mutations present at a low rate of 0.125%. However, due to the loss of molecules, the sensitivity is slightly low and the error rate is high compared to the amplicon sequencing protocol. Therefore, the amplicon sequencing protocol-based SPIDER-seq method becomes a better option in terms of ctDNA loss rather than the capture method when starting with a low number of molecules.

TABLE S1
Read numbers, UID pairs, CID and barcodes
used in the present invention.

| | KP | QM | PH |
|---|---|---|---|
| Trimmed pair-reads | 17,379,861 | 36,596,076 | 50,555,163 |
| UID pairs | 1,280,164 | 2,249,912 | 2,205,754 |
| UID pairs used | 88,982 | 138,857 | 96,023 |
| CIDs obtained | 54,780 | 89,684 | 61,789 |
| Content number | 2,788 | 3,488 | 2,500 |

TABLE S2
Baseline distribution of BRAF p.V600 gene loci. Each base was
calculated with the original data and consensus sequence based on CID and UID.

| | | Variant | | | | | |
|---|---|---|---|---|---|---|---|
| | | allele | | | | | |
| | | frequency | | | | | |
| Position | Identifier | (%) | Replicate | A | T | C | G |
| chr7: 140753336 | CID | 0 | 1 | 21,022 | 0 | 0 | 0 |
| | | | 2 | 29,543 | 0 | 0 | 0 |
| | | | 3 | 14,851 | 4 | 0 | 0 |
| | | 0.125 | 1 | 73,231 | 42 | 0 | 0 |
| | | | 2 | 54,982 | 64 | 0 | 0 |
| | | | 3 | 58,233 | 40 | 0 | 0 |
| | | 0.5 | 1 | 66,444 | 357 | 0 | 0 |
| | | | 2 | 43,077 | 165 | 0 | 0 |
| | | | 3 | 40,253 | 190 | 0 | 0 |
| | | 1 | 1 | 26,562 | 193 | 0 | 0 |
| | | | 2 | 36,186 | 273 | 0 | 1 |
| | | | 3 | 47,226 | 585 | 0 | 10 |
| | UID | 0 | 1 | 104,362 | 6 | 0 | 15 |
| | | | 2 | 142,637 | 9 | 0 | 7 |
| | | | 3 | 79,264 | 27 | 1 | 3 |
| | | 0.125 | 1 | 390,582 | 202 | 1 | 36 |
| | | | 2 | 281,317 | 312 | 2 | 28 |
| | | | 3 | 331,802 | 294 | 2 | 34 |
| | | 0.5 | 1 | 328,924 | 1,764 | 1 | 37 |
| | | | 2 | 213,003 | 831 | 1 | 25 |
| | | | 3 | 194,821 | 960 | 0 | 15 |
| | | 1 | 1 | 150,478 | 1,186 | 5 | 12 |
| | | | 2 | 177,528 | 1,377 | 1 | 12 |
| | | | 3 | 252,068 | 3,214 | 1 | 58 |
| | No identifier | 0 | 1 | 251,660 | 34 | 1 | 38 |
| | (Raw data) | | 2 | 372,229 | 64 | 8 | 28 |
| | | | 3 | 185,022 | 87 | 4 | 17 |
| | | 0.125 | 1 | 1169,451 | 680 | 10 | 151 |
| | | | 2 | 837,196 | 1,022 | 213 | 108 |
| | | | 3 | 734,586 | 713 | 5 | 107 |
| | | 0.5 | 1 | 795,916 | 4,335 | 66 | 101 |
| | | | 2 | 599,347 | 2,461 | 7 | 90 |
| | | | 3 | 518,203 | 2,600 | 2 | 72 |
| | | | 1 | 319,169 | 2,556 | 13 | 40 |
| | | 1 | 2 | 458,381 | 3,632 | 10 | 64 |
| | | | 3 | 798,129 | 10,239 | 11 | 241 |

TABLE S3
Baseline distribution of BRAF p.V600 peripheral
positions in CID-based consensus sequences.

| | Variant | | | | | |
|---|---|---|---|---|---|---|
| | allele | | | | | |
| | frequency | | | | | |
| Position | (%) | Replicate | A | T | C | G |
| chr7: | 0 | 1 | 0 | 21,028 | 0 | 0 |
| 140753332 | | 2 | 0 | 29,549 | 0 | 0 |
| | | 3 | 0 | 14,854 | 0 | 0 |
| | 0.125 | 1 | 0 | 73,279 | 0 | 0 |
| | | 2 | 0 | 55,048 | 0 | 0 |
| | | 3 | 0 | 58,288 | 0 | 0 |
| | 0.5 | 1 | 0 | 66,811 | 0 | 0 |
| | | 2 | 0 | 43,246 | 0 | 0 |
| | | 3 | 0 | 40,445 | 0 | 0 |
| | 1 | 1 | 0 | 26,763 | 0 | 0 |
| | | 2 | 0 | 36,470 | 0 | 0 |
| | | 3 | 0 | 47,831 | 0 | 0 |
| chr7: | 0 | 1 | 0 | 21,027 | 0 | 0 |
| 140753333 | | 2 | 0 | 29,545 | 0 | 0 |
| | | 3 | 0 | 14,850 | 0 | 0 |
| | 0.125 | 1 | 0 | 73,270 | 0 | 0 |
| | | 2 | 0 | 55,049 | 0 | 0 |
| | | 3 | 0 | 58,269 | 0 | 0 |
| | 0.5 | 1 | 0 | 66,795 | 0 | 0 |
| | | 2 | 0 | 43,239 | 0 | 0 |
| | | 3 | 0 | 40,435 | 0 | 0 |
| | 1 | 1 | 0 | 26,760 | 0 | 0 |
| | | 2 | 0 | 36,463 | 0 | 0 |
| | | 3 | 0 | 47,813 | 0 | 0 |
| chr7: | 0 | 1 | 0 | 21,017 | 4 | 0 |
| 140753334 | | 2 | 0 | 29,548 | 0 | 0 |
| | | 3 | 0 | 14,855 | 0 | 0 |
| | 0.125 | 1 | 0 | 73,280 | 0 | 0 |
| | | 2 | 0 | 55,044 | 0 | 0 |
| | | 3 | 0 | 58,275 | 0 | 0 |
| | 0.5 | 1 | 0 | 66,811 | 0 | 0 |
| | | 2 | 0 | 43,252 | 0 | 0 |
| | | 3 | 0 | 40,446 | 0 | 0 |
| | 1 | 1 | 0 | 26,762 | 0 | 0 |
| | | 2 | 0 | 36,457 | 0 | 0 |
| | | 3 | 0 | 47,823 | 0 | 0 |
| chr7: | 0 | 1 | 0 | 0 | 21,024 | 0 |
| 140753335 | | 2 | 0 | 0 | 29,547 | 0 |
| | | 3 | 6 | 0 | 14,850 | 0 |
| | 0.125 | 1 | 3 | 0 | 73,278 | 0 |
| | | 2 | 38 | 0 | 55,015 | 0 |
| | | 3 | 9 | 6 | 58,268 | 0 |
| | 0.5 | 1 | 14 | 1 | 66,800 | 0 |
| | | 2 | 0 | 0 | 43,251 | 0 |
| | | 3 | 0 | 0 | 40,446 | 0 |
| | 1 | 1 | 0 | 0 | 26,755 | 0 |
| | | 2 | 30 | 0 | 36,425 | 0 |
| | | 3 | 0 | 1 | 47,828 | 0 |
| chr7: | 0 | 1 | 10 | 0 | 21,013 | 0 |
| 140753337 | | 2 | 0 | 0 | 29,548 | 0 |
| | 0.125 | 3 | 0 | 0 | 14,850 | 0 |
| | | 1 | 0 | 2 | 73,276 | 1 |
| | | 2 | 0 | 0 | 55,046 | 0 |
| | | 3 | 0 | 0 | 58,281 | 0 |
| | 0.5 | 1 | 0 | 4 | 66,802 | 0 |
| | | 2 | 15 | 0 | 43,241 | 0 |
| | | 3 | 0 | 12 | 40,434 | 0 |
| | 1 | 1 | 0 | 0 | 26,766 | 0 |
| | | 2 | 0 | 0 | 36,464 | 0 |
| | | 3 | 0 | 0 | 47,823 | 0 |
| chr7: | 0 | 1 | 0 | 21,020 | 1 | 0 |
| 140753338 | | 2 | 0 | 29,545 | 0 | 0 |
| | | 3 | 0 | 14,857 | 0 | 0 |
| | 0.125 | 1 | 0 | 73,271 | 0 | 0 |
| | | 2 | 0 | 55,036 | 0 | 0 |
| | | 3 | 0 | 58,272 | 0 | 0 |
| | 0.5 | 1 | 0 | 66,791 | 10 | 0 |
| | | 2 | 0 | 43,250 | 1 | 0 |
| | | 3 | 0 | 40,445 | 0 | 0 |
| | 1 | 1 | 0 | 26,759 | 0 | 0 |
| | | 2 | 0 | 36,463 | 0 | 0 |
| | | 3 | 0 | 47,828 | 0 | 0 |
| chr7: | 0 | 1 | 0 | 0 | 0 | 21,025 |
| 140753339 | | 2 | 24 | 0 | 0 | 29,519 |
| | | 3 | 0 | 0 | 0 | 14,858 |
| | 0.125 | 1 | 8 | 0 | 0 | 73,268 |
| | | 2 | 0 | 0 | 0 | 55,047 |
| | | 3 | 0 | 0 | 0 | 58,265 |
| | 0.5 | 1 | 0 | 11 | 0 | 66,794 |
| | | 2 | 2 | 17 | 0 | 43,231 |
| | | 3 | 0 | 0 | 0 | 40,447 |
| | 1 | 1 | 0 | 11 | 0 | 26,749 |
| | | 2 | 1 | 0 | 0 | 36,461 |
| | | 3 | 0 | 21 | 0 | 47,807 |
| chr7: | 0 | 1 | 0 | 21,026 | 0 | 0 |
| 140753340 | | 2 | 0 | 29,548 | 0 | 0 |
| | | 3 | 0 | 14,857 | 0 | 0 |
| | 0.125 | 1 | 0 | 73,275 | 4 | 0 |
| | | 2 | 0 | 55,049 | 0 | 0 |
| | | 3 | 0 | 58,282 | 0 | 0 |
| | 0.5 | 1 | 0 | 66,814 | 0 | 0 |
| | | 2 | 0 | 43,251 | 0 | 0 |
| | | 3 | 0 | 40,440 | 0 | 0 |
| | 1 | 1 | 0 | 26,758 | 0 | 0 |
| | | 2 | 0 | 36,461 | 0 | 0 |
| | | 3 | 0 | 47,829 | 0 | 0 |

TABLE S4
List of targets for multiplex PCR experiments.

| | Mutation | | Mutation position | | Amplicon |
|---|---|---|---|---|---|
| Target | type | HGVS_Nomenclature | (GRCH38) | Strand | size |
| NRAS(p.Q61R) | Substitution | c.182A > G | chr1: 114713908 | − | 78 |
| KRAS(p.G12D) | Substitution | c.35G > A | chr12: 25245350 | − | 81 |
| CTNNB1(p.T41A) | Substitution | c.121A > G | chr3: 41224633 | + | 77 |
| JAK2(p.V617F) | Substitution | c.1849G > T | chr9: 5073770 | + | 90 |
| PDGFRA(p.D842V) | Substitution | c.2525A > T | chr4: 54285926 | + | 100 |
| PIK3CA | Substitution | c.3140A > G | chr3: 179234297 | + | 74 |
| (p.H1047R) | | | | | |
| EGFR(p.T790M) | Substitution | c.2369C > T | chr7: 55181378 | + | 106 |
| EGFR(p.L858R) | Substitution | c.2573T > G | chr7: 55191822 | + | 76 |
| BRAF(p.V600E) | Substitution | c.1799T > A | chr7: 140753336 | − | 94 |
| EGFR | Deletion | c.2236_2250del15 | chr7: 55174773-55174787 | + | 89 |
| (p.E746_A750del ELREA) | | | | | |

TABLE S5
List of targets for hybridization capture.

| | Mutation | | Mutation position | |
|---|---|---|---|---|
| Target | type | HGVS_nomenclature | (GRCH38) | Strand |
| NRAS-p.Q61R | Substitution | c.182A > G | Chr1: 114713908 | − |
| RET-p.M918T | Substitution | c.2753T > C | chr10: 43121968 | + |
| ATM-p.C353fs*5 | Deletion | c.1058_1059delGT | chr11: 108247120-108247121 | + |
| KRAS-p.G12D | Substitution | c.35G > A | chr12: 25245350 | − |
| FLT3-p.D835Y | Substitution | c.2503G > T | chr13: 28018505 | − |
| AKT1-p.E17K | Substitution | c.49G > A | chr14: 104780214 | − |
| ERBB2-p.A775_G776insYVMA | Insertion | c.2324_2325ins12 | chr17: 39724742-39724743 | + |
| TP53-p.R175H | Substitution | c.524G > A | chr17: 7675088 | − |
| TP53-p.R248Q | Substitution | c.743G > A | chr17: 7674220 | − |
| TP53-p.R273H | Substitution | c.818G > A | chr17: 7673802 | − |
| GNA11-p.Q209L | Substitution | c.626A > T | chr19: 3118944 | + |
| IDH1-p.R132C | Substitution | c.394C > T | chr2: 208248389 | − |
| GNAS-p.R201C | Substitution | c.601C > T | chr20: 58909365 | + |
| CTNNB1-p.T41A | Substitution | c.121A > G | 41224633 | + |
| FOXL2-p.C134W | Substitution | c.402C > G | chr3: 138946321 | − |
| PIK3CA-p.E545K | Substitution | c.1633G > A | chr3: 179218303 | + |
| PIK3CA-p.H1047R | Substitution | c.3140A > G | chr3: 179234297 | + |
| FGFR3-p.S249C | Substitution | c.746C > G | chr4: 1801841 | + |
| KIT-p.D816V | Substitution | c.2447A > T | chr4: 54733155 | + |
| PDGFRA-p.D842V | Substitution | c.2525A > T | chr4: 54285926 | + |
| APC-p.R1450* | Substitution | c.4348C > T | chr5: 112839942 | + |
| EGFR-p.E746_A750delELREA | Deletion | c.2236_2250del15 | chr7: 55174773-55174787 | + |
| EGFR-p.D770_N771insG | Insertion | c.2310_2311insGGT | chr7: 55181319-55181320 | + |
| EGFR-p.L858R | Substitution | c.2573T > G | chr7: 55191822 | + |
| BRAF-p.V600E | Substitution | c.1799T > A | chr7: 140753336 | − |
| EGFR-p.T790M | Substitution | c.2369C > T | chr7: 55181378 | + |
| GNAQ-p.Q209P | Substitution | c.626A > C | chr9: 77794572 | − |
| JAK2-p.V617F | Substitution | c.1849G > T | chr9: 5073770 | + |

TABLE S6
Coverage for each experiment.

| | Variant Allele | | | | |
|---|---|---|---|---|---|
| Replicate | Frequency (%) | replicate 1 | replicate 2 | replicate 3 | replicate 4 |
| AKT1-p.E17K | 0 | 385087 | 290282 | 435919 | 411243 |
| APC-p.R1450* | | 271004 | 204143 | 323543 | 326981 |
| ATM-p.C353fs*5 | | 266194 | 196108 | 274922 | 280229 |
| BRAF-p.V600E | | 326257 | 232642 | 310381 | 322605 |
| CTNNB1-p.T41A | | 577006 | 432372 | 605078 | 612902 |
| EGFR-p.D770_N771insG | | 670323 | 548045 | 653662 | 688472 |
| EGFR-p.E746_A750delELREA | | 235825 | 180339 | 260573 | 258897 |
| EGFR-p.L858R | | 752832 | 563805 | 690438 | 777531 |
| EGFR-p.T790M | | 742562 | 615392 | 739939 | 770818 |
| ERBB2-p.A775_G776insYVMA | | 715691 | 580868 | 832360 | 902435 |
| FGFR3-p.S249C | | 51687 | 40553 | 46463 | 51604 |
| FLT3-p.D835Y | | 434036 | 323959 | 415116 | 418313 |
| FOXL2-p.C134W | | 88443 | 78974 | 73363 | 80827 |
| GNA11-p.Q209L | | 550798 | 453012 | 648473 | 639805 |
| GNAQ-p.Q209P | | 324003 | 270423 | 309730 | 335105 |
| GNAS-p.R201C | | 273720 | 216435 | 293799 | 325356 |
| IDH1-p.R132C | | 369479 | 276122 | 361381 | 376629 |
| JAK2-p.V617F | | 402254 | 303246 | 370567 | 371570 |
| KIT-p.D816V | | 417346 | 330100 | 414802 | 448430 |
| KRAS-p.G12D | | 493407 | 349848 | 418577 | 466475 |
| NRAS-p.Q61R | | 306714 | 219640 | 267041 | 282955 |
| PDGFRA-p.D842V | | 500706 | 368601 | 531649 | 517931 |
| PIK3CA-p.E545K | | 44778 | 35115 | 38926 | 44164 |
| PIK3CA-p.H1047R | | 433206 | 327090 | 434958 | 478961 |
| RET-p.M918T | | 346406 | 279298 | 338412 | 335418 |
| TP53-p.R175H | | 834909 | 607283 | 751572 | 822903 |
| TP53-p.R248Q | | 763062 | 601957 | 826733 | 811083 |
| TP53-p.R273H | | 497425 | 390444 | 495590 | 509962 |
| AKT1-p.E17K | 0.125 | 291818 | 358964 | 458123 | 353622 |
| APC-p.R1450* | | 177596 | 230609 | 276249 | 210585 |
| ATM-p.C353fs*5 | | 148836 | 132578 | 179457 | 137295 |
| BRAF-p.V600E | | 200284 | 155054 | 184913 | 169534 |
| CTNNB1-p.T41A | | 410072 | 421662 | 538555 | 437159 |
| EGFR-p.D770_N771insG | | 517063 | 598588 | 792973 | 611236 |
| EGFR-p.E746_A750delELREA | | 156402 | 191311 | 240321 | 182118 |
| EGFR-p.L858R | | 474021 | 542134 | 647430 | 507515 |
| EGFR-p.T790M | | 595735 | 643170 | 855499 | 641082 |
| ERBB2-p.A775_G776insYVMA | | 541722 | 649415 | 850544 | 680777 |
| FGFR3-p.S249C | | 43897 | 48860 | 62164 | 48843 |
| FLT3-p.D835Y | | 297980 | 310725 | 376299 | 308177 |
| FOXL2-p.C134W | | 63544 | 70176 | 99121 | 73442 |
| GNA11-p.Q209L | | 418689 | 497786 | 622246 | 561045 |
| GNAQ-p.Q209P | | 198962 | 176543 | 213609 | 173550 |
| GNAS-p.R201C | | 207709 | 223026 | 280587 | 226198 |
| IDH1-p.R132C | | 266667 | 240992 | 285963 | 245869 |
| JAK2-p.V617F | | 237045 | 197116 | 238961 | 203728 |
| KIT-p.D816V | | 258938 | 221485 | 278536 | 226706 |
| KRAS-p.G12D | | 295642 | 258166 | 316254 | 263426 |
| NRAS-p.Q61R | | 220865 | 207561 | 231387 | 209334 |
| PDGFRA-p.D842V | | 323232 | 380752 | 477192 | 375221 |
| PIK3CA-p.E545K | | 19987 | 18325 | 20301 | 18068 |
| PIK3CA-p.H1047R | | 279231 | 265601 | 323312 | 269603 |
| RET-p.M918T | | 223554 | 254192 | 304633 | 243818 |
| TP53-p.R175H | | 600662 | 680827 | 880584 | 666725 |
| TP53-p.R248Q | | 606878 | 715176 | 832819 | 708169 |
| TP53-p.R273H | | 348103 | 365668 | 455495 | 338875 |
| AKT1-p.E17K | 0.25 | 392849 | 110609 | 243588 | 409311 |
| APC-p.R1450* | | 297012 | 82005 | 240738 | 331858 |
| ATM-p.C353fs*5 | | 258058 | 74308 | 215021 | 315330 |
| BRAF-p.V600E | | 282463 | 83040 | 236819 | 343286 |
| CTNNB1-p.T41A | | 556474 | 153841 | 432184 | 598725 |
| EGFR-p.D770_N771insG | | 631933 | 184620 | 430576 | 700095 |
| EGFR-p.E746_A750delELREA | | 260333 | 82380 | 210421 | 312823 |
| EGFR-p.L858R | | 703631 | 194464 | 483343 | 758842 |
| EGFR-p.T790M | | 674471 | 196891 | 469134 | 730536 |
| ERBB2-p.A775_G776insYVMA | | 704764 | 203187 | 498778 | 756048 |
| FGFR3-p.S249C | | 55940 | 17963 | 30196 | 62708 |
| FLT3-p.D835Y | | 366447 | 103213 | 292675 | 425740 |
| FOXL2-p.C134W | | 98497 | 24573 | 55586 | 87501 |
| GNA11-p.Q209L | | 654086 | 176323 | 411163 | 686187 |
| GNAQ-p.Q209P | | 246198 | 76766 | 234460 | 332367 |
| GNAS-p.R201C | | 305811 | 82901 | 225473 | 346336 |
| IDH1-p.R132C | | 356785 | 106840 | 305187 | 420183 |
| JAK2-p.V617F | | 351406 | 101303 | 295524 | 441442 |
| KIT-p.D816V | | 377283 | 107499 | 322499 | 450291 |
| KRAS-p.G12D | | 375774 | 101249 | 316712 | 414388 |
| NRAS-p.Q61R | | 245353 | 68050 | 200976 | 271175 |
| PDGFRA-p.D842V | | 507389 | 135498 | 372308 | 560502 |
| PIK3CA-p.E545K | | 41348 | 12061 | 37015 | 50746 |
| PIK3CA-p.H1047R | | 368311 | 108111 | 332804 | 473388 |
| RET-p.M918T | | 297379 | 92500 | 244429 | 376182 |
| TP53-p.R175H | | 719675 | 196514 | 478048 | 795687 |
| TP53-p.R248Q | | 726627 | 209101 | 515337 | 794057 |
| TP53-p.R273H | | 460993 | 128856 | 342250 | 527136 |
| AKT1-p.E17K | 0.5 | 464440 | 219039 | 399452 | 477427 |
| APC-p.R1450* | | 335243 | 130947 | 258774 | 283888 |
| ATM-p.C353fs*5 | | 235149 | 113863 | 202403 | 230748 |
| BRAF-p.V600E | | 287184 | 138486 | 250961 | 282152 |
| CTNNB1-p.T41A | | 657466 | 285125 | 540398 | 589719 |
| EGFR-p.D770_N771insG | | 815756 | 373080 | 657086 | 750294 |
| EGFR-p.E746_A750delELREA | | 275097 | 117067 | 253407 | 272599 |
| EGFR-p.L858R | | 726918 | 396019 | 694977 | 755262 |
| EGFR-p.T790M | | 888161 | 418082 | 710061 | 820762 |
| ERBB2-p.A775_G776insYVMA | | 821922 | 418613 | 758721 | 828054 |
| FGFR3-p.S249C | | 60397 | 28499 | 60684 | 65622 |
| FLT3-p.D835Y | | 464201 | 220534 | 391916 | 426451 |
| FOXL2-p.C134W | | 112889 | 52105 | 82014 | 93911 |
| GNA11-p.Q209L | | 710920 | 353351 | 622261 | 660159 |
| GNAQ-p.Q209P | | 291534 | 135744 | 258966 | 281788 |
| GNAS-p.R201C | | 320095 | 156726 | 272577 | 311993 |
| IDH1-p.R132C | | 363335 | 194396 | 352872 | 385544 |
| JAK2-p.V617F | | 355087 | 168133 | 296777 | 332601 |
| KIT-p.D816V | | 391680 | 191215 | 324847 | 368422 |
| KRAS-p.G12D | | 418328 | 209253 | 363548 | 397659 |
| NRAS-p.Q61R | | 278688 | 148294 | 251401 | 275948 |
| PDGFRA-p.D842V | | 554472 | 247443 | 479176 | 538187 |
| PIK3CA-p.E545K | | 34384 | 18428 | 27464 | 32864 |
| PIK3CA-p.H1047R | | 392546 | 201238 | 335435 | 407163 |
| RET-p.M918T | | 343749 | 170832 | 303805 | 355676 |
| TP53-p.R175H | | 918946 | 447491 | 785769 | 899555 |
| TP53-p.R248Q | | 861374 | 440565 | 797500 | 903357 |
| TP53-p.R273H | | 526072 | 250382 | 464640 | 538217 |
| AKT1-p.E17K | 1 | 188185 | 264210 | 365880 | 346579 |
| APC-p.R1450* | | 161316 | 255094 | 289174 | 277891 |
| ATM-p.C353fs*5 | | 130400 | 185553 | 243775 | 254193 |
| BRAF-p.V600E | | 154927 | 222349 | 279540 | 268400 |
| CTNNB1-p.T41A | | 316912 | 440898 | 547876 | 563574 |
| EGFR-p.D770_N771insG | | 331354 | 499108 | 616120 | 596998 |
| EGFR-p.E746_A750delELREA | | 152286 | 218903 | 264943 | 233773 |
| EGFR-p.L858R | | 352950 | 547447 | 644850 | 606492 |
| EGFR-p.T790M | | 355540 | 534454 | 661214 | 637232 |
| ERBB2-p.A775_G776insYVMA | | 348986 | 540811 | 663555 | 631434 |
| FGFR3-p.S249C | | 21882 | 28292 | 43569 | 36833 |
| FLT3-p.D835Y | | 205494 | 310281 | 395321 | 356008 |
| FOXL2-p.C134W | | 41022 | 57483 | 85841 | 74818 |
| GNA11-p.Q209L | | 283654 | 368656 | 502975 | 490103 |
| GNAQ-p.Q209P | | 158219 | 217845 | 292753 | 262694 |
| GNAS-p.R201C | | 161962 | 227938 | 305396 | 271843 |
| IDH1-p.R132C | | 214241 | 314317 | 379620 | 367287 |
| JAK2-p.V617F | | 183674 | 265174 | 328553 | 334642 |
| KIT-p.D816V | | 211608 | 313664 | 380049 | 362399 |
| KRAS-p.G12D | | 217651 | 307948 | 406711 | 386587 |
| NRAS-p.Q61R | | 165336 | 213936 | 263044 | 261314 |
| PDGFRA-p.D842V | | 272141 | 384092 | 474566 | 484680 |
| PIK3CA-p.E545K | | 18982 | 26832 | 33639 | 34570 |
| PIK3CA-p.H1047R | | 234991 | 345097 | 437220 | 407976 |
| RET-p.M918T | | 188629 | 269911 | 328189 | 310859 |
| TP53-p.R175H | | 388012 | 531587 | 666912 | 606570 |
| TP53-p.R248Q | | 414160 | 532470 | 676403 | 668171 |
| TP53-p.R273H | | 252855 | 376991 | 453221 | 407341 |

TABLE S7
Number of consensus reads per experiment.

| | Variant allele | | | | |
|---|---|---|---|---|---|
| Replicate | frequency (%) | rep 1 | rep2 | rep3 | rep4 |
| AKT1-p.E17K | 0 | 3712 | 6069 | 4504 | 2402 |
| APC-p.R1450* | | 2371 | 3477 | 4480 | 2778 |
| ATM-p.C353fs*5 | | 2246 | 3241 | 4102 | 2600 |
| BRAF-p.V600E | | 2675 | 3996 | 4096 | 2623 |
| CTNNB1-p.T41A | | 5971 | 8663 | 6252 | 3995 |
| EGFR-p.D770_N771insG | | 10694 | 18057 | 9728 | 8908 |
| EGFR-p.E746_A750delELREA | | 1615 | 2928 | 3212 | 1936 |
| EGFR-p.L858R | | 8444 | 11915 | 5062 | 3152 |
| EGFR-p.T790M | | 6902 | 11925 | 5671 | 3456 |
| ERBB2-p.A775_G776insYVMA | | 6436 | 9987 | 6549 | 3877 |
| FLT3-p.D835Y | | 3820 | 5547 | 5191 | 3124 |
| GNA11-p.Q209L | | 5485 | 8855 | 5717 | 3518 |
| GNAQ-p.Q209P | | 2717 | 4412 | 4564 | 3094 |
| GNAS-p.R201C | | 2292 | 4328 | 4263 | 2863 |
| IDH1-p.R132C | | 3271 | 4818 | 4752 | 3006 |
| JAK2-p.V617F | | 3678 | 5288 | 4749 | 2991 |
| KIT-p.D816V | | 3790 | 6331 | 5005 | 3369 |
| KRAS-p.G12D | | 4391 | 6321 | 5386 | 3723 |
| NRAS-p.Q61R | | 2986 | 4491 | 3606 | 2302 |
| PDGFRA-p.D842V | | 4574 | 6665 | 5005 | 3054 |
| PIK3CA-p.H1047R | | 3414 | 5649 | 5429 | 3678 |
| RET-p.M918T | | 2959 | 5131 | 4067 | 2366 |
| TP53-p.R175H | | 7598 | 11545 | 5733 | 3412 |
| TP53-p.R248Q | | 8372 | 12089 | 6732 | 3951 |
| TP53-p.R273H | | 5489 | 8573 | 4562 | 2729 |
| AKT1-p.E17K | 0.125 | 2754 | 12390 | 13531 | 9725 |
| APC-p.R1450* | | 1320 | 9631 | 10122 | 7687 |
| ATM-p.C353fs*5 | | 1239 | 6136 | 6719 | 5100 |
| BRAF-p.V600E | | 1548 | 6666 | 6733 | 5901 |
| CTNNB1-p.T41A | | 3977 | 16247 | 17678 | 12908 |
| EGFR-p.D770_N771insG | | 8022 | 28439 | 30457 | 22404 |
| EGFR-p.E746_A750delELREA | | 982 | 7370 | 7928 | 5850 |
| EGFR-p.L858R | | 5091 | 14222 | 14472 | 10879 |
| EGFR-p.T790M | | 5335 | 16845 | 18123 | 13157 |
| ERBB2-p.A775_G776insYVMA | | 4771 | 17509 | 18621 | 13692 |
| FLT3-p.D835Y | | 2487 | 11985 | 12541 | 9773 |
| GNA11-p.Q209L | | 4042 | 14758 | 15963 | 12635 |
| GNAQ-p.Q209P | | 1608 | 8102 | 8219 | 6391 |
| GNAS-p.R201C | | 1581 | 9896 | 10846 | 8397 |
| IDH1-p.R132C | | 2289 | 9829 | 10070 | 8126 |
| JAK2-p.V617F | | 2118 | 8388 | 8672 | 6879 |
| KIT-p.D816V | | 2281 | 9256 | 9780 | 7482 |
| KRAS-p.G12D | | 2650 | 11358 | 11706 | 8775 |
| NRAS-p.Q61R | | 1987 | 9185 | 8895 | 7380 |
| PDGFRA-p.D842V | | 2729 | 12704 | 13194 | 9788 |
| PIK3CA-p.H1047R | | 2106 | 10610 | 10952 | 8795 |
| RET-p.M918T | | 1728 | 10033 | 10598 | 7873 |
| TP53-p.R175H | | 5066 | 17591 | 18706 | 13839 |
| TP53-p.R248Q | | 6375 | 20340 | 21520 | 16063 |
| TP53-p.R273H | | 3591 | 12006 | 12911 | 9367 |
| AKT1-p.E17K | 0.25 | 5601 | 5788 | 3611 | 5923 |
| APC-p.R1450* | | 3569 | 4716 | 4485 | 6463 |
| ATM-p.C353fs*5 | | 2972 | 4517 | 4208 | 6561 |
| BRAF-p.V600E | | 3302 | 4623 | 4260 | 6441 |
| CTNNB1-p.T41A | | 7863 | 8690 | 6589 | 10049 |
| EGFR-p.D770_N771insG | | 13955 | 14115 | 9438 | 15281 |
| EGFR-p.E746_A750delELREA | | 2780 | 4368 | 3604 | 5471 |
| EGFR-p.L858R | | 9779 | 8683 | 5127 | 7913 |
| EGFR-p.T790M | | 8975 | 8523 | 5432 | 8524 |
| ERBB2-p.A775_G776insYVMA | | 8949 | 9577 | 5731 | 8994 |
| FLT3-p.D835Y | | 4341 | 5811 | 5084 | 7530 |
| GNA11-p.Q209L | | 8920 | 8525 | 5578 | 9115 |
| GNAQ-p.Q209P | | 2759 | 4666 | 4652 | 6843 |
| GNAS-p.R201C | | 3876 | 4989 | 4477 | 7110 |
| IDH1-p.R132C | | 4385 | 6166 | 5480 | 7819 |
| JAK2-p.V617F | | 4266 | 6126 | 5234 | 8294 |
| KIT-p.D816V | | 4808 | 6130 | 5560 | 7984 |
| KRAS-p.G12D | | 4621 | 6227 | 5873 | 7977 |
| NRAS-p.Q61R | | 3348 | 4289 | 3809 | 5317 |
| PDGFRA-p.D842V | | 6392 | 6923 | 5284 | 8222 |
| PIK3CA-p.H1047R | | 4205 | 6022 | 5588 | 8499 |
| RET-p.M918T | | 3672 | 5245 | 4152 | 6612 |
| TP53-p.R175H | | 9549 | 8340 | 5433 | 8634 |
| TP53-p.R248Q | | 10456 | 9925 | 6247 | 10446 |
| TP53-p.R273H | | 6785 | 6609 | 4915 | 7290 |
| AKT1-p.E17K | 0.5 | 4688 | 5114 | 7776 | 9929 |
| APC-p.R1450* | | 3130 | 2211 | 6700 | 8074 |
| ATM-p.C353fs*5 | | 2058 | 2048 | 5376 | 7019 |
| BRAF-p.V600E | | 2425 | 2541 | 5959 | 7521 |
| CTNNB1-p.T41A | | 6898 | 6577 | 11785 | 14003 |
| EGFR-p.D770_N771insG | | 13586 | 14158 | 19283 | 23017 |
| EGFR-p.E746_A750delELREA | | 1980 | 1919 | 5877 | 7074 |
| EGFR-p.L858R | | 7729 | 9411 | 10270 | 11468 |
| EGFR-p.T790M | | 8456 | 9299 | 11213 | 13213 |
| ERBB2-p.A775_G776insYVMA | | 7624 | 8527 | 11859 | 13735 |
| FLT3-p.D835Y | | 4024 | 4127 | 8802 | 10831 |
| GNA11-p.Q209L | | 6808 | 7754 | 10163 | 11756 |
| GNAQ-p.Q209P | | 2485 | 2273 | 6819 | 8422 |
| GNAS-p.R201C | | 2752 | 3189 | 6931 | 9197 |
| IDH1-p.R132C | | 3183 | 3696 | 8401 | 10215 |
| JAK2-p.V617F | | 3269 | 3011 | 7018 | 8485 |
| KIT-p.D816V | | 3658 | 3921 | 7295 | 9090 |
| KRAS-p.G12D | | 3855 | 4014 | 8950 | 10771 |
| NRAS-p.Q61R | | 2734 | 3069 | 6321 | 7699 |
| PDGFRA-p.D842V | | 5214 | 4955 | 9293 | 11393 |
| PIK3CA-p.H1047R | | 3230 | 3613 | 7473 | 10228 |
| RET-p.M918T | | 3077 | 3229 | 6877 | 8843 |
| TP53-p.R175H | | 8736 | 10164 | 11559 | 13338 |
| TP53-p.R248Q | | 9132 | 10265 | 12809 | 15163 |
| TP53-p.R273H | | 5733 | 6055 | 8824 | 10315 |
| AKT1-p.E17K | 1 | 4679 | 4683 | 5717 | 8747 |
| APC-p.R1450* | | 3161 | 4069 | 4288 | 7417 |
| ATM-p.C353fs*5 | | 2832 | 2938 | 3748 | 6576 |
| BRAF-p.V600E | | 3238 | 3313 | 3958 | 6849 |
| CTNNB1-p.T41A | | 8250 | 8519 | 9432 | 14878 |
| EGFR-p.D770_N771insG | | 7480 | 7952 | 8379 | 13431 |
| EGFR-p.E746_A750delELREA | | 2652 | 2833 | 3197 | 5321 |
| EGFR-p.L858R | | 9839 | 10896 | 10033 | 14802 |
| EGFR-p.T790M | | 8658 | 9219 | 9551 | 14096 |
| ERBB2-p.A775_G776insYVMA | | 8493 | 9256 | 9725 | 13981 |
| FLT3-p.D835Y | | 4466 | 4942 | 6029 | 9015 |
| GNA11-p.Q209L | | 7549 | 6775 | 8543 | 12939 |
| GNAQ-p.Q209P | | 3388 | 3516 | 4216 | 6872 |
| GNAS-p.R201C | | 3363 | 3558 | 4723 | 7724 |
| IDH1-p.R132C | | 4824 | 5213 | 5732 | 9281 |
| JAK2-p.V617F | | 4329 | 4442 | 4882 | 8414 |
| KIT-p.D816V | | 5096 | 5551 | 5897 | 9247 |
| KRAS-p.G12D | | 4934 | 5111 | 6235 | 10007 |
| NRAS-p.Q61R | | 4198 | 3766 | 4535 | 7567 |
| PDGFRA-p.D842V | | 6417 | 6488 | 6889 | 11578 |
| PIK3CA-p.H1047R | | 4882 | 5083 | 6119 | 9705 |
| RET-p.M918T | | 4159 | 4351 | 4686 | 7790 |
| TP53-p.R175H | | 8976 | 8836 | 9287 | 13540 |
| TP53-p.R248Q | | 11787 | 10500 | 11478 | 17187 |
| TP53-p.R273H | | 6993 | 7339 | 7399 | 10534 |

TABLE 8
[Table S8] Oligonucleotides used in the present invention.

| | |
|---|---|
| Name | Sequence |

In case of denatured barcode (or UID) content

| | |
|---|---|
| BRAF_ | ACTGTTTTCCTTTACTTACTACACCTCAGATATATTTCTTCATGAAGACCTCACAGT |
| N12 | AAAAATAGGTGANNNNNNTCTAGCTACAGAGAAATCTCGATNNNNNNGGTCCCATC |
| | AGTTTGAACAGTTGTCTGGATCCATTTTGTGGATGGTAAGAATTGAGGCTATTTTTCC |
| | AC |

Primary amplification primers (UID tagging amplification)

| | |
|---|---|
| NRAS_ | CACTCTTTCCCTACACGACGCTCTTCCGATCTTCGGTCACTTAGGANNNNANNNN |
| Q61_ | GNNNNCNNNNATAGATGGTGAAACCTGTTTGTTGG |
| P5 | |
| KRAS_ | CACTCTTTCCCTACACGACGCTCTTCCGATCTCGAGAGTTGGATGCTNNNNTNNN |
| G12_ | NANNNNGNNNNTATTATAAGGCCTGCTGAAAATG |
| P5 | |
| CTNNB1_ | CACTCTTTCCCTACACGACGCTCTTCCGATCTGCATCAATGCCGTCANNNNCNNN |
| T41_ | NTNNNNANNNNCAACAGTCTTACCTGGACTCTGG |
| P5 | |
| JAK2_ | CACTCTTTCCCTACACGACGCTCTTCCGATCTAGGTGGCGAACCTNNNNGNNNN |
| V617_ | CNNNNTNNNNAAGCTTTCTCACAAGCATTTGGTTT |
| P5 | |
| PDGFRA_ | CACTCTTTCCCTACACGACGCTCTTCCGATCTTGCACTAACGATCCANNNNANNN |
| D842_ | NGNNNNCNNNNGCACAAGGAAAAATTGTGAAGAT |
| P5 | |
| PIK3CA- | CACTCTTTCCCTACACGACGCTCTTCCGATCTCTCACTCCTCCAGTCNNNNCNNN |
| 1047_ | NTNNNNANNNNAACTGAGCAAGAGGCTTTGG |
| P5 | |
| PIK3CA- | CACTCTTTCCCTACACGACGCTCTTCCGATCTTGAGCAGTGTCTTGNNNNGNNNN |
| 545_ | CNNNNTNNNNGCTCAAAGCAATTTCTACACGAGAT |
| P5 | |
| EGFR- | CACTCTTTCCCTACACGACGCTCTTCCGATCTCACTTACTCCGAACCNNNNANNN |
| 790_ | NGNNNNCNNNNGCAGGTACTGGGAGCCAAT |
| P5 | |
| EGFR- | CACTCTTTCCCTACACGACGCTCTTCCGATCTCAGAAGTGTGTGAGCNNNNANN |
| 858_ | NNGNNNNCNNNNGCAGCATGTCAAGATCACAGATT |
| P5 | |
| EGFR_ | CACTCTTTCCCTACACGACGCTCTTCCGATCTCTTCAACTGATAGCGNNNNTNNN |
| ex19_ | NANNNNGNNNNGAAAGTTAAAATTCCCGTCGCTAT |
| P5 | |
| BRAF- | CACTCTTTCCCTACACGACGCTCTTCCGATCTGACTTGTTCAGGATTNNNNTNNN |
| v600_ | NANNNNGNNNNTGAAGACCTCACAGTAAAAATAG |
| P5 | |
| NRAS_ | GACTGGAGTTCAGACGTGTGCTCTTCCGATCTAACGAGGTCTACTTCNNNNANN |
| Q61_ | NNGNNNNCNNNNATGTATTGGTCTCTCATGGCA |
| P7 | |
| KRAS_ | GACTGGAGTTCAGACGTGTGCTCTTCCGATCTGAACCGTACTCGTTCNNNNTNN |
| G12_ | NNANNNNGNNNNTATCGTCAAGGCACTCTT |
| P7 | |
| CTNNB1_ | GACTGGAGTTCAGACGTGTGCTCTTCCGATCTGGCTTAAGGATCCAGNNNNCNN |
| T41_ | NNTNNNNANNNNCAGGATTGCCTTTACCACTCA |
| P7 | |
| JAK2_ | GACTGGAGTTCAGACGTGTGCTCTTCCGATCTCCAGTCAGTGCTCNNNNGNNNN |
| V617_ | CNNNNTNNNNAGAAAGGCATTAGAAAGCCTGTAGTT |
| P7 | |
| PDGFRA | GACTGGAGTTCAGACGTGTGCTCTTCCGATCTGAGAAGTTGCTCGAGNNNNANN |
| D842_ | NNGNNNNCNNNNAGGGAAGTGAGGACGTACACTG |
| P7 | |
| PIK3CA- | GACTGGAGTTCAGACGTGTGCTCTTCCGATCTGCTTGTCTGAGTAGTNNNNCNN |
| 1047_ | NNTNNNNANNNNCATTTTTGTTGTCCAGCCACC |
| P7 | |
| PIK3CA- | GACTGGAGTTCAGACGTGTGCTCTTCCGATCTGGATTGTTCAANNNNGNNNNCN |
| 545_ | NNNTNNNNTGTCTGTGACTCCATAGAAAATCTTTCT |
| P7 | |
| EGFR- | GACTGGAGTTCAGACGTGTGCTCTTCCGATCTCCATAGAGAACCAACNNNNTNN |
| 790_ | NNANNNNGNNNNGCATCTGCCTCACCTCCA |
| P7 | |
| EGFR- | GACTGGAGTTCAGACGTGTGCTCTTCCGATCTAGTGTATGGATACCNNNNANNNN |
| 858_ | GNNNNCNNNNCCTCCTTCTGCATGGTATTCTTTCT |
| P7 | |
| EGFR_ | GACTGGAGTTCAGACGTGTGCTCTTCCGATCTTGCAAGTCGTAGACTNNNNTNN |
| ex19_ | NNANNNNGNNNNAAAGCAGAAACTCACATCGA |
| P7 | |
| BRAF- | GACTGGAGTTCAGACGTGTGCTCTTCCGATCTTAGGTATCCTAAGCGNNNNTNNN |
| v600_ | NANNNNGNNNNATGGATCCAGACAACTGTTC |
| P7 | |

Primers for amplifying hybridization capture library

| | |
|---|---|
| NEB | AATGATACGGCGACCACCGAGATCTACACGGCNNNNNACACTCTTTCCCTACAC |
| Next-i5- | GACGCTCTTCCGATC*T |
| N5_1 | |
| NEB | AATGATACGGCGACCACCGAGATCTACACTCTNNNNNACACTCTTTCCCTACACG |
| Next-i5- | ACGCTCTTCCGATC*T |
| N5_2 | |
| NEB | AATGATACGGCGACCACCGAGATCTACACCTANNNNNACACTCTTTCCCTACACG |
| Next-i5- | ACGCTCTTCCGATC*T |
| N5_3 | |
| NEB | AATGATACGGCGACCACCGAGATCTACACAAGNNNNNACACTCTTTCCCTACAC |
| Next-i5- | GACGCTCTTCCGATC*T |
| N5_4 | |
| NEB | CAAGCAGAAGACGGCATACGAGATTTGNNNNNGTGACTGGAGTTCAGACGTGT |
| Next-i7- | GCTCTTCCGATC*T |
| N5_1 | |
| NEB | CAAGCAGAAGACGGCATACGAGATGGTNNNNNGTGACTGGAGTTCAGACGTGT |
| Next-i7- | GCTCTTCCGATC*T |
| N5_2 | |
| NEB | CAAGCAGAAGACGGCATACGAGATCACNNNNNGTGACTGGAGTTCAGACGTGT |
| Next-i7- | GCTCTTCCGATC*T |
| N5_3 | |
| NEB | CAAGCAGAAGACGGCATACGAGATACANNNNNGTGACTGGAGTTCAGACGTGT |
| Next-i7- | GCTCTTCCGATC*T |
| N5_4 | |

Sequences indicated in bold represent random bases (N = A, T, C or G) and asterisks indicate phosphorothioate bonds.

TABLE S9
Materials used in the present invention

| | | | |
|---|---|---|---|
| Product Name | Product No. | Supplier | Description |

cfDNA reference genomic DNA

| | | | |
|---|---|---|---|
| Seraseq ™ ctDNA Mutation | 0710-0144 | SeraCare | ctDNA model |
| Mix v2 WT | | Life Sciences | (Human, AF = 0%) |
| Seraseq ™ ctDNA Mutation | 0710-0143 | SeraCare | ctDNA model (Human, |
| Mix v2 AF0.125% | | Life Sciences | AF = 0.125%) |
| Seraseq ™ ctDNA Mutation | 0710-0142 | SeraCare | ctDNA model (Human, |
| Mix v2 AF0.25% | | Life Sciences | AF = 0.25%) |
| Seraseq ™ ctDNA Mutation | 0710-0141 | SeraCare | ctDNA model (Human, |
| Mix v2 AF0.5% | | Life Sciences | AF = 0.5%) |
| Seraseq ™ ctDNA Mutation | 0710-0141 | SeraCare | ctDNA model (Human, |
| Mix v2 AF1% | | Life Sciences | AF = 1%) |

Polymerases

| | | | |
|---|---|---|---|
| HotStart PCR Kit, with dNTPs | 07958897001 | Roche | KAPA HiFi polymerase |
| | | | 2x master mix contains 4 ul |
| | | | of 5X KAPA HiFi Buffer 0.6 ul |
| | | | of 10 mM KAPA dNTP Mix, 0.4 |
| | | | ul of KAPA HiFi HotStart DNA |
| | | | Polymerase |
| Phusion High-Fidelity DNA | M0530S | NEB | Phusion polymerase |
| Polymerase | | | |
| QIAGEN Multiplex PCR Kit | 206143 | QIAGEN | Qiagen multiplex Taq |
| | | | polymerase |

Purification

| | | | |
|---|---|---|---|
| AMPure XP | A63881 | BECKMAN | PCR cleanup kit for |
| | | COULTER | hybridization capture library |
| MinElute Gel Extraction Kit | 28606 | QIAGEN | Purification kit of amplicon |
| | | | library |

Enzymes for hybridization capture library preparation

| | | | |
|---|---|---|---|
| 5X ER/A-Tailing Enzyme Mix | Y9420L | Enzymatics | Enzyme mix for end repair |
| | | | and A tailing reaction |
| WGS ligase | L6030-W-L | Enzymatics | Ligation of NGS adaptor |
| USER Enzyme | M5505S | NEB | Cleavage of Uracil in the |
| | | | NEBNext adaptor |

TABLE S10
Amounts of cfDNA reference standards used in the present
invention. (hGE = haploid genome equivalent)

| | | | BRAF | | 11-gene | | Hybridization | |
|---|---|---|---|---|---|---|---|---|
| | | | targeting | | targeting | | capture | |
| | | Concentration | experiment | | experiment | | experiment | |
| Product Name | Description | (ng/ul) | ng | hGEs | ng | hGEs | ng | hGEs |
| Seraseq ™ | ctDNA model | 15.6 | 15.6 | 4727 | 31.2 | 9455 | 31.2 | 9455 |
| ctDNA Mutation | (Human, | | | | | | | |
| Mix v2 WT | AF = 0%) | | | | | | | |
| Seraseq ™ | ctDNA model | 15.8 | 15.8 | 4788 | 31.6 | 9576 | 31.6 | 9576 |
| ctDNA Mutation | (Human, | | | | | | | |
| Mix v2 AF0.125% | AF = 0.125%) | | | | | | | |
| Seraseq ™ | ctDNA model | 13.9 | Not | Not | 27.8 | 8424 | 27.8 | 8424 |
| ctDNA Mutation | (Human, | | used | used | | | | |
| Mix v2 AF0.25% | AF = 0.25%) | | | | | | | |
| Seraseq ™ | ctDNA model | 14.8 | 14.8 | 4485 | Not | Not | 29.6 | 8970 |
| ctDNA Mutation | (Human, | | | | used | used | | |
| Mix v2 AF0.5% | AF = 0.5%) | | | | | | | |
| Seraseq ™ | ctDNA model | | | | Not | Not | | |
| ctDNA Mutation | (Human, | 12.2 | 12.2 | 3697 | used | used | 24.4 | 7394 |
| Mix v2 AF1% | AF = 1%) | | | | | | | |

The above-described description of the present invention is provided for illustrative purposes, and those skilled in the art to which the present invention pertains will understand that the present invention can be easily modified into other specific forms without changing the technical spirit or essential features of the present invention. Therefore, it should be understood that the above-described embodiments are only exemplary in all aspects and are not restrictive. Furthermore, the scope of the present invention is represented by the following claims, and it should be interpreted that the meaning and scope of the claims and all the changes or modified forms derived from the equivalent concepts thereof fall within the scope of the present invention.

What is claimed is:

1. A method for generating a consensus sequence for detecting a target nucleic acid, the method comprising: amplifying DNA fragments from a sample using polymerase chain reaction (PCR) with primers containing adapter sequences, flanking sequences, and UID sequences, in the direction from the 5′ end to the 3′ end;
obtaining sequence information of the amplified DNA fragments through the PCR; and
generating a cluster using a peer-to-peer (P2P) network method based on the obtained sequence information.

2. The method of claim 1, wherein the adapter sequence is 17 bp to 69 bp long.

3. The method of claim 1, further comprising a step of trimming the sequence information of the amplified DNA fragments through the PCR.

4. The method of claim 1, wherein the UID sequence consists of 12 to 25 random nucleic acids.

5. The method of claim 4, wherein the UID sequence comprises repeats of N and X in the form (N)m(X)n,
wherein N is a random base, X is a fixed base, and
m is a constant from 2 to 5, and n is a constant from 1 to 2.

6. The method of claim 1, wherein the PCR is performed for 3 to 8 cycles.

7. The method of claim 1, wherein the P2P network method is an algorithm method comprising: obtaining the sequence information of a UID pair from the sequence information of the amplified DNA fragments through the PCR;
grouping a second UID including first UID sequence information and grouping a first UID including second UID sequence information among the sequence information of the obtained UID pairs; and
selecting one UID sequence from the grouping of the second UID or the grouping of the first UID, and then connecting a UID sequence pair selected from the unselected UID groups.

8. The method of claim 1, wherein the cluster is a group comprising molecules derived from the same molecule formed by the P2P network method.

9. The method of claim 1, wherein the DNA of the sample is ctDNA.

10. A kit for generating a consensus sequence for detecting a target nucleic acid, comprising a PCR primer comprising adapter sequences, a flanking sequence and a UID sequence.


SEQUENCE LISTING (31 sequences)

SEQ ID NO: 1 - protein, 173 aa, Artificial Sequence
  BRAF_N12
  PEPTIDE (1)..(173): BRAF_N12
ACTGTTTTCCTTTACTTACTACACCTCAGATATATTTCTTCATGAAGACCTCACAGTAAAAATAGGTGANNNNNNTCTAGCTACAGAGAAATCTCGATNNNNNNGGTCCCATCAGTTTGAACAGTTGTCTGGATCCATTTTGTGGATGGTAAGAATTGAGGCTATTTTTCCAC

SEQ ID NO: 2 - protein, 90 aa, Artificial Sequence
  NRAS_Q61_P5
  PEPTIDE (1)..(90): NRAS_Q61_P5
CACTCTTTCCCTACACGACGCTCTTCCGATCTTCGGTCACTTAGGANNNNANNNNGNNNNCNNNNATAGATGGTGAAACCTGTTTGTTGG

SEQ ID NO: 3 - protein, 89 aa, Artificial Sequence
  KRAS_G12_P5
  PEPTIDE (1)..(89): KRAS_G12_P5
CACTCTTTCCCTACACGACGCTCTTCCGATCTCGAGAGTTGGATGCTNNNNTNNNNANNNNGNNNNTATTATAAGGCCTGCTGAAAATG

SEQ ID NO: 4 - protein, 89 aa, Artificial Sequence
  CTNNB1_T41_P5
  PEPTIDE (1)..(89): CTNNB1_T41_P5
CACTCTTTCCCTACACGACGCTCTTCCGATCTGCATCAATGCCGTCANNNNCNNNNTNNNNANNNNCAACAGTCTTACCTGGACTCTGG

SEQ ID NO: 5 - protein, 89 aa, Artificial Sequence
  JAK2_V617_P5
  PEPTIDE (1)..(89): JAK2_V617_P5
CACTCTTTCCCTACACGACGCTCTTCCGATCTAGGTGGCGAACCTNNNNGNNNNCNNNNTNNNNAAGCTTTCTCACAAGCATTTGGTTT

SEQ ID NO: 6 - protein, 89 aa, Artificial Sequence
  PDGFRA_D842_P5
  PEPTIDE (1)..(89): PDGFRA_D842_P5
CACTCTTTCCCTACACGACGCTCTTCCGATCTTGCACTAACGATCCANNNNANNNNGNNNNCNNNNGCACAAGGAAAAATTGTGAAGAT

SEQ ID NO: 7 - protein, 86 aa, Artificial Sequence
  PIK3CA-1047_P5
  PEPTIDE (1)..(86): PIK3CA-1047_P5
CACTCTTTCCCTACACGACGCTCTTCCGATCTCTCACTCCTCCAGTCNNNNCNNNNTNNNNANNNNAACTGAGCAAGAGGCTTTGG

SEQ ID NO: 8 - protein, 90 aa, Artificial Sequence
  PIK3CA-545_P5
  PEPTIDE (1)..(90): PIK3CA-545_P5
CACTCTTTCCCTACACGACGCTCTTCCGATCTTGAGCAGTGTCTTGNNNNGNNNNCNNNNTNNNNGCTCAAAGCAATTTCTACACGAGAT

SEQ ID NO: 9 - protein, 85 aa, Artificial Sequence
  EGFR-790_P5
  PEPTIDE (1)..(85): EGFR-790_P5
CACTCTTTCCCTACACGACGCTCTTCCGATCTCACTTACTCCGAACCNNNNANNNNGNNNNCNNNNGCAGGTACTGGGAGCCAAT

SEQ ID NO: 10 - protein, 89 aa, Artificial Sequence
  EGFR-858_P5
  PEPTIDE (1)..(89): EGFR-858_P5
CACTCTTTCCCTACACGACGCTCTTCCGATCTCAGAAGTGTGTGAGCNNNNANNNNGNNNNCNNNNGCAGCATGTCAAGATCACAGATT

SEQ ID NO: 11 - protein, 90 aa, Artificial Sequence
  EGFR_ex19_P5
  PEPTIDE (1)..(90): EGFR_ex19_P5
CACTCTTTCCCTACACGACGCTCTTCCGATCTCTTCAACTGATAGCGNNNNTNNNNANNNNGNNNNGAAAGTTAAAATTCCCGTCGCTAT

SEQ ID NO: 12 - protein, 89 aa, Artificial Sequence
  BRAF-v600_P5
  PEPTIDE (1)..(89): BRAF-v600_P5
CACTCTTTCCCTACACGACGCTCTTCCGATCTGACTTGTTCAGGATTNNNNTNNNNANNNNGNNNNTGAAGACCTCACAGTAAAAATAG

SEQ ID NO: 13 - protein, 87 aa, Artificial Sequence
  NRAS_Q61_P7
  PEPTIDE (1)..(87): NRAS_Q61_P7
GACTGGAGTTCAGACGTGTGCTCTTCCGATCTAACGAGGTCTACTTCNNNNANNNNGNNNNCNNNNATGTATTGGTCTCTCATGGCA

SEQ ID NO: 14 - protein, 84 aa, Artificial Sequence
  KRAS_G12_P7
  PEPTIDE (1)..(84): KRAS_G12_P7
GACTGGAGTTCAGACGTGTGCTCTTCCGATCTGAACCGTACTCGTTCNNNNTNNNNANNNNGNNNNTATCGTCAAGGCACTCTT

SEQ ID NO: 15 - protein, 87 aa, Artificial Sequence
  CTNNB1_T41_P7
  PEPTIDE (1)..(87): CTNNB1_T41_P7
GACTGGAGTTCAGACGTGTGCTCTTCCGATCTGGCTTAAGGATCCAGNNNNCNNNNTNNNNANNNNCAGGATTGCCTTTACCACTCA

SEQ ID NO: 16 - protein, 90 aa, Artificial Sequence
  JAK2_V617_P7
  PEPTIDE (1)..(90): JAK2_V617_P7
GACTGGAGTTCAGACGTGTGCTCTTCCGATCTCCAGTCAGTGCTCNNNNGNNNNCNNNNTNNNNAGAAAGGCATTAGAAAGCCTGTAGTT

SEQ ID NO: 17 - protein, 88 aa, Artificial Sequence
  PDGFRA_D842_P7
  PEPTIDE (1)..(88): PDGFRA_D842_P7
GACTGGAGTTCAGACGTGTGCTCTTCCGATCTGAGAAGTTGCTCGAGNNNNANNNNGNNNNCNNNNAGGGAAGTGAGGACGTACACTG

SEQ ID NO: 18 - protein, 87 aa, Artificial Sequence
  PIK3CA-1047_P7
  PEPTIDE (1)..(87): PIK3CA-1047_P7
GACTGGAGTTCAGACGTGTGCTCTTCCGATCTGCTTGTCTGAGTAGTNNNNCNNNNTNNNNANNNNCATTTTTGTTGTCCAGCCACC

SEQ ID NO: 19 - protein, 90 aa, Artificial Sequence
  PIK3CA-545_P7
  PEPTIDE (1)..(90): PIK3CA-545_P7
GACTGGAGTTCAGACGTGTGCTCTTCCGATCTGGATTGTTCAANNNNGNNNNCNNNNTNNNNTGTCTGTGACTCCATAGAAAATCTTTCT

SEQ ID NO: 20 - protein, 84 aa, Artificial Sequence
  EGFR-790_P7
  PEPTIDE (1)..(84): EGFR-790_P7
GACTGGAGTTCAGACGTGTGCTCTTCCGATCTCCATAGAGAACCAACNNNNTNNNNANNNNGNNNNGCATCTGCCTCACCTCCA

SEQ ID NO: 21 - protein, 90 aa, Artificial Sequence
  EGFR-858_P7
  PEPTIDE (1)..(90): EGFR-858_P7
GACTGGAGTTCAGACGTGTGCTCTTCCGATCTAGTGTATGGATACCNNNNANNNNGNNNNCNNNNCCTCCTTCTGCATGGTATTCTTTCT

SEQ ID NO: 22 - protein, 86 aa, Artificial Sequence
  EGFR_ex19_P7
  PEPTIDE (1)..(86): EGFR_ex19_P7
GACTGGAGTTCAGACGTGTGCTCTTCCGATCTTGCAAGTCGTAGACTNNNNTNNNNANNNNGNNNNAAAGCAGAAACTCACATCGA

SEQ ID NO: 23 - protein, 86 aa, Artificial Sequence
  BRAF-v600_P7
  PEPTIDE (1)..(86): BRAF-v600_P7
GACTGGAGTTCAGACGTGTGCTCTTCCGATCTTAGGTATCCTAAGCGNNNNTNNNNANNNNGNNNNATGGATCCAGACAACTGTTC

SEQ ID NO: 24 - protein, 70 aa, Artificial Sequence
  NEBNext-i5-N5_1
  PEPTIDE (1)..(70): NEBNext-i5-N5_1
AATGATACGGCGACCACCGAGATCTACACGGCNNNNNACACTCTTTCCCTACACGACGCTCTTCCGATCT

SEQ ID NO: 25 - protein, 70 aa, Artificial Sequence
  NEBNext-i5-N5_2
  PEPTIDE (1)..(70): NEBNext-i5-N5_2
AATGATACGGCGACCACCGAGATCTACACTCTNNNNNACACTCTTTCCCTACACGACGCTCTTCCGATCT

SEQ ID NO: 26 - protein, 70 aa, Artificial Sequence
  NEBNext-i5-N5_3
  PEPTIDE (1)..(70): NEBNext-i5-N5_3
AATGATACGGCGACCACCGAGATCTACACCTANNNNNACACTCTTTCCCTACACGACGCTCTTCCGATCT

SEQ ID NO: 27 - protein, 70 aa, Artificial Sequence
  NEBNext-i5-N5_4
  PEPTIDE (1)..(70): NEBNext-i5-N5_4
AATGATACGGCGACCACCGAGATCTACACAAGNNNNNACACTCTTTCCCTACACGACGCTCTTCCGATCT

SEQ ID NO: 28 - protein, 66 aa, Artificial Sequence
  NEBNext-i7-N5_1
  PEPTIDE (1)..(66): NEBNext-i7-N5_1
CAAGCAGAAGACGGCATACGAGATTTGNNNNNGTGACTGGAGTTCAGACGTGTGCTCTTCCGATCT

SEQ ID NO: 29 - protein, 66 aa, Artificial Sequence
  NEBNext-i7-N5_2
  PEPTIDE (1)..(66): NEBNext-i7-N5_2
CAAGCAGAAGACGGCATACGAGATGGTNNNNNGTGACTGGAGTTCAGACGTGTGCTCTTCCGATCT

SEQ ID NO: 30 - protein, 66 aa, Artificial Sequence
  NEBNext-i7-N5_3
  PEPTIDE (1)..(66): NEBNext-i7-N5_3
CAAGCAGAAGACGGCATACGAGATCACNNNNNGTGACTGGAGTTCAGACGTGTGCTCTTCCGATCT

SEQ ID NO: 31 - protein, 66 aa, Artificial Sequence
  NEBNext-i7-N5_4
  PEPTIDE (1)..(66): NEBNext-i7-N5_4
CAAGCAGAAGACGGCATACGAGATACANNNNNGTGACTGGAGTTCAGACGTGTGCTCTTCCGATCT